(19) United States
(12) Patent Application Publication
(10) Pub. No.: US 2023/0128672 A1
(43) Pub. Date: Apr. 27, 2023

(54) Methods and Systems for Measuring Progesterone Metabolites

(71) Applicant: Laboratory Corporation of America Holdings, Burlington, NC (US)

(72) Inventors: Brett Holmquist, Thousand Oaks, CA (US); Mary Katherine Morr Kelemen, Thousand Oaks, CA (US)

(21) Appl. No.: 17/975,288

(22) Filed: Oct. 27, 2022

Related U.S. Application Data
(60) Provisional application No. 63/272,517, filed on Oct. 27, 2021.

(51) Int. Cl. G01N 33/68; G16H 10/40

(52) U.S. Cl. CPC G01N 33/689; G01N 33/6848; G16H 10/40

(57) ABSTRACT

Disclosed are methods and systems using liquid chromatography-tandem mass spectrometry (LC-MS/MS) for the detection and/or analysis of progesterone metabolites, such as progesterone sulfates, in biological samples. In some cases, the amount of progesterone sulfate may be used to distinguish whether gestational pruritus of the skin is an early symptom of (ICP) or due to benign pruritus gravidarum.

### RELATED APPLICATIONS

This application claims priority to U.S. Provisional Application No. 63/272,517 filed on Oct. 27, 2021. The entire content of said provisional application is herein incorporated by reference for all purposes.

### FIELD

The presently disclosed subject matter relates to methods and systems for the analysis of progesterone metabolite biomarkers. In certain embodiments, the biomarker measurement may be used for clinical diagnosis.

### BACKGROUND

Biomarkers, such as hormones, vitamins, and metabolites can be used for screening or diagnosis of certain disorders. For example, various metabolites of progesterone can be important indicators of various physiological states such as menopause and breast cancer. Additionally, progesterone metabolites may play a role in ovulation and the ability to maintain a healthy pregnancy. Thus, a challenge in obstetrics is to distinguish pathological symptoms from those associated with normal changes of pregnancy. Progesterone metabolites, including certain progesterone sulfates, may be good indicators of the efficacy of various therapeutic compounds to reduce complications of pregnancy such as Intrahepatic Cholestasis of Pregnancy (ICP) and complications that can occur therefrom such as prenatal death, preterm delivery, and/or iatrogenic preterm delivery. For example, in some cases it is important to differentiate whether gestational pruritus of the skin is an early symptom of ICP or due to benign pruritus gravidarum (Hayyeh et al., Hepatology 63:1287-1298 (2016)). ICP is characterized by raised serum bile acids and complicated by spontaneous preterm labor and stillbirth. A biomarker for ICP would be invaluable for early diagnosis and treatment and to enable its differentiation from other maternal diseases. Measurement of progesterone sulfates can enable targeted obstetric care to a high-risk population.

Thus, there is a need to develop analytical techniques that can be used for the measurement of progesterone metabolites.

### SUMMARY

In some embodiments, disclosed is a method for determining the presence or amount of a metabolite of progesterone in a sample by mass spectrometry. In an embodiment, tandem mass spectrometry (MS/MS) is used. The method may comprise the steps of: (a) generating one or more precursor ions from a progesterone metabolite; (b) generating one or more product ions of the one or more precursor ions; and (c) detecting the presence or amount of the one or more precursor ions generated in step (a) or the one or more product ions of step (b) or both. In an embodiment, the detected ions are used to determine the amount of the progesterone metabolite in the sample. In certain embodiments, the progesterone metabolite is a progesterone sulfate. In certain embodiments, the mass spectrometry is tandem mass spectrometry.

Also disclosed are systems for performing the methods or any of the steps of the methods disclosed herein. In certain embodiments, the system may comprise: a station and/or component for providing a sample; optionally, a station and/or component for partially purifying a progesterone metabolite from other components in the sample; optionally, a station and/or component for chromatographically separating the progesterone metabolite from other components in the sample; a station and/or component for mass spectrometry to generate one or more precursor ions and one or more product ions from the progesterone metabolite; and a station and/or component to analyze the mass spectrum to determine the presence or amount of the progesterone metabolite in the test sample. In some embodiments, certain of the stations or components are combined as single stations or components. In certain embodiments, the progesterone metabolite is a progesterone sulfate. In certain embodiments, at least one of the stations may be controlled by a computer.

Also disclosed are computer program products tangibly embodied in a non-transitory machine-readable storage medium, including instructions configured to run the disclosed systems or any part (e.g., component) of the disclosed systems and/or perform a step or steps of any of the disclosed methods.

### BRIEF DESCRIPTION OF THE DRAWINGS

The present disclosure may be better understood by reference to the following non-limiting figures.

FIG. 1 shows a flow chart of a method for quantitative analysis of a progesterone metabolite in accordance with one embodiment of the disclosure.

FIG. 2 shows a system for quantitative analysis of a progesterone metabolite in accordance with one embodiment of the disclosure.

FIG. 3 shows an exemplary computing device in accordance with various embodiments of the disclosure.

FIG. 4 shows a PM3S multiple reaction monitoring (MRM) chromatogram (10 pairs) showing the analyte peak for the 399.40→97.100 transition using Turbo Spray LC-MS/MS in accordance with certain embodiments of the disclosure.

FIG. 5 shows a PM4S and PM5S MRM chromatogram showing the analyte peak for the 397.3→97.0 transition for PM5S and the analyte peak for the 397.2→97.0 transition for PM4S in accordance with certain embodiments of the disclosure.

FIG. 6 shows a PM2DiS and PM3DiS MRM chromatogram showing the analyte peak for the sum of 479.098→381.2+479.099→381.2+479.100→381.2+479.101→381.2+479.102→381.2 transitions for PM2DiS and the analyte peak for the sum of the 479.098→399.1+479.099→399.1+479.100→399.1+479.101→399.1+479.102→399.1 transitions for PM3DiS in accordance with certain embodiments of the disclosure.

### DETAILED DESCRIPTION

The disclosed subject matter now will be described more fully hereinafter with reference to the accompanying description and drawings, in which some, but not all embodiments of the disclosed subject matter are shown. The disclosed subject matter can be embodied in many different forms and should not be construed as limited to the embodiments set forth herein. Like numbers refer to like elements throughout.

Many modifications and other embodiments of the disclosed subject matter set forth herein will come to mind to one skilled in the art to which the disclosed subject matter pertains having the benefit of the teachings presented in the descriptions and the associated drawings. Therefore, it is to be understood that the presently disclosed subject matter is not to be limited to the specific embodiments disclosed herein and that modifications and other embodiments are intended to be included within the scope of the appended claims. Although specific terms are employed herein, they are used in a generic and descriptive sense only and not for purposes of limitation. . Additionally, any reference referred to as being “incorporated herein” is to be understood as being incorporated in its entirety.

### Definitions and Descriptions

While the following terms are believed to be well understood by one of ordinary skill in the art, the following definitions are set forth to facilitate explanation of the presently disclosed subject matter. Other definitions are found throughout the specification. Unless otherwise defined, all technical and scientific terms used herein have the same meaning as commonly understood by one of ordinary skill in the art to which this presently described subject matter belongs.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

The terms “a”, “an”, and “the” refer to “one or more” when used in this application, including the claims. Thus, for example, reference to “a cell” includes a plurality of such cells, unless the context clearly is to the contrary (e.g., a plurality of cells), and so forth.

As used herein, “AMR” or “Analytical Measurement Range” is the range of analyte values that a method can directly measure on the specimen without any dilution, concentration, or other pretreatment not part of the usual assay process.

As used herein, the term “analytical column” refers to a chromatography column having sufficient chromatographic plates to effect a separation of the components of a test sample matrix. Preferably, the components eluted from the analytical column are separated in such a way to allow the presence or amount of an analyte(s) of interest to be determined. In some embodiments, the analytical column comprises particles having an average diameter of about 5 p.m or less. In some embodiments, the analytical column is a functionalized silica or polymer-silica hybrid, or a polymeric particle or monolithic silica stationary phase, such as a phenyl-hexyl functionalized analytical column. Analytical columns can be distinguished from “extraction columns,” which typically are used to separate or extract retained materials from non-retained materials to obtain a “purified” sample for further purification or analysis.

As used herein “analyte” is a component represented in the name of a measurable quantity.

As used herein “analytic interference” refers to an artifactual increase or decrease in apparent concentrations, activity, or intensity of an analyte due to the presence of a substance that reacts specifically or nonspecifically with either the detection reagent or the signal itself.

The term “Atmospheric Pressure Chemical Ionization” or “APCI” as used herein refers to mass spectroscopy methods produce ions by ion-molecule reactions that occur within a plasma at atmospheric pressure. The plasma is maintained by an electric discharge between the spray capillary and a counter electrode. Then, ions are typically extracted into a mass analyzer by use of a set of differentially pumped skimmer stages. A counterflow of dry and preheated N₂ gas may be used to improve removal of solvent. The gas-phase ionization in APCI can be more effective than ESI for analyzing less-polar species.

The term “Atmospheric Pressure Photoionization” or “APPI” as used herein refers to the form of mass spectroscopy where the mechanism for the photoionization of molecule M is photon absorption and electron ejection to form the molecular M+. Because the photon energy typically is just above the ionization potential, the molecular ion is less susceptible to dissociation. In many cases it may be possible to analyze samples without the need for chromatography, thus saving significant time and expense. In the presence of water vapor or protic solvents, the molecular ion can extract H to form MH+. This tends to occur if M has a high proton affinity. This does not affect quantitation accuracy because the sum of M+ and MH+ is constant. Compounds in protic solvents are usually observed as MH+, whereas nonpolar compounds usually form M+ (see e.g., Robb et al., Anal. Chem. 72(15): 3653-3659 (2000)).

As used herein, the term “biological sample” refers to a sample obtained from a biological source, including, but not limited to, an animal, a cell culture, an organ culture, and the like. Suitable samples include cell-free DNA, blood, plasma, serum, urine, saliva, tear, nasopharyngeal swabs, cerebrospinal fluid, organ, hair, muscle, or other tissue samples.

The term “chemical ionization” as used herein refers to methods in which a reagent gas (e.g. ammonia) is subjected to electron impact, and analyte ions are formed by the interaction of reagent gas ions and analyte molecules.

As used herein, “chromatography” refers to a process in which a chemical mixture carried by a liquid or gas is separated into components as a result of differential distribution of the chemical entities as they flow around or over a stationary liquid or solid phase.

The term “electron ionization” as used herein refers to methods in which an analyte of interest in a gaseous or vapor phase interacts with a flow of electrons. Impact of the electrons with the analyte produces analyte ions, which may then be subjected to a mass spectrometry technique.

The term “electrospray ionization” or “ESI” as used herein refers to methods in which a solution is passed along a short length of capillary tube, to the end of which is applied a high positive or negative electric potential. Upon reaching the end of the tube, the solution may be vaporized (nebulized) into a jet or spray of very small droplets of solution in solvent vapor. This mist of droplet can flow through an evaporation chamber which is heated slightly to prevent condensation and to evaporate solvent. As the droplets get smaller the electrical surface charge density increases until such time that the natural repulsion between like charges causes ions as well as neutral molecules to be released.

The term “field desorption” as used herein refers to methods in which a non-volatile test sample is placed on an ionization surface, and an intense electric field is used to generate analyte ions.

As used herein, the term “HPLC” or “high performance liquid chromatography” refers to liquid chromatography in which the degree of separation is increased by forcing the mobile phase under pressure through a stationary phase, typically a densely packed column. The chromatographic column typically includes a medium (i.e., a packing material) to facilitate separation of chemical moieties (i.e., fractionation). The medium may include minute particles. The particles include a bonded surface that interacts with the various chemical moieties to facilitate separation of the chemical moieties such as the biomarker analytes quantified in the experiments herein. One suitable bonded surface is a hydrophobic bonded surface such as an alkyl bonded surface. Alkyl bonded surfaces may include C-4, C-8, or C-18 bonded alkyl groups, preferably C-18 bonded groups. The chromatographic column includes an inlet port for receiving a sample and an outlet port for discharging an effluent that includes the fractionated sample. In the method, the sample (or pre-purified sample) may be applied to the column at the inlet port, eluted with a solvent or solvent mixture, and discharged at the outlet port. Different solvent modes may be selected for eluting different analytes of interest. For example, liquid chromatography may be performed using a gradient mode, an isocratic mode, or a polytyptic (i.e. mixed) mode.

The term “ionization” and “ionizing” as used herein refers to the process of generating an analyte ion having a net electrical charge equal to one or more electron units. Negative ions are those ions having a net negative charge of one or more electron units, while positive ions are those ions having a net positive charge of one or more electron units.

As used herein, the term “ion summing” or “signal summing” refers to the practice of summing discrete chromatograms (e.g., tandem mass spectrometry) of essentially identical transitions in a manner to increase the signal to noise ratio. By programming multiple transitions in the same cycle time, the dwell time for each individual transition is diminished, although the signal will be approximately the same intensity for each transition, allowing for summing of the signals. As noise is random, the summation of replicates of the signal will yield an approximately linear increase in signal, while random noise will diminish (see e.g., Pauwels et al., Anal. Bioanal. Chem., 407:6191-6199 (2015)).

As used herein, “interference” refers to the negative influence of the presence of non-analytes in a sample, e.g., due to hemolysis, lipemia, and/or icterus, to be able to accurately measure an analyte.

As used herein, “liquid chromatography” or “LC” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid (i.e., mobile phase), as this fluid moves relative to the stationary phase(s). Liquid chromatography includes reverse phase liquid chromatography (RPLC), high performance liquid chromatography (HPLC) and high turbulence liquid chromatography (HTLC).

As used herein, “LOD” or “Limit of Detection” is the lowest amount of analyte in a sample that can be detected with stated probability. Typically, LOD is expressed as the limit of blank (LOB) plus 1.645×SD (or 2×SD) of low sample measurements. Also, as used herein, “LLOQ” or “Lower Limit of Quantitation” is the lowest amount of analyte in a sample that can be quantitatively determined with stated acceptable precision and accuracy.

The term “matrix-assisted laser desorption ionization” or “MALDI” as used herein refers to methods in which a non-volatile sample is exposed to laser irradiation, which desorbs and ionizes analytes in the sample by various ionization pathways, including photo-ionization, protonation, deprotonation, and cluster decay. For MALDI, the sample is mixed with an energy-absorbing matrix, which facilitates desorption of analyte molecules.

The terms “mass spectrometry” or “MS” as used herein generally refer to methods of filtering, detecting, and measuring ions based on their mass-to-charge ratio, or “m/z.” In MS techniques, one or more molecules of interest are ionized, and the ions are subsequently introduced into a mass spectrometer where, due to a combination of electric fields, the ions follow a path in space that is dependent upon mass (“m”) and charge (“z”). The term “tandem mass spectrometry” or “MS/MS” refers to a type of mass spectrometry whereby a molecule is ionized in a first step to form a parent (or precursor) ions and these ions are separated by their mass to charge (m/z) ratio. Parent ions of a particular m/z are then selected and fragmented to form daughter (or product or fragment) ions and the daughter ions are then separated by their m/z ratio. Triple quadrupole mass spectrometers use the first and third quadrupoles as mass filters and the second quadrupole for fragmentation, e.g., by collision-induced dissociation, ionization or other techniques discussed herein.

As used herein, the term “on-line” refers to purification or separation steps that are performed in such a way that the test sample is disposed, e.g., injected, into a system in which the various components of the system are operationally connected and, in some embodiments, in fluid communication with one another. In contrast to the term on-line, the term “off-line” refers to a purification, separation, or extraction procedure that is performed separately from previous and/or subsequent purification or separation steps and/or analysis steps.

As used herein, “precision” is expressed as standard deviation (SD) and/or percent coefficient of variation (% CV). “Intra-run precision” is the closeness of the agreement between the results of successive measurements of the same measure and carried under the same conditions of measurements (same analytical run). “Inter-run precision” is the closeness of the agreement between independent test results obtained under stipulated conditions (different analytical runs and/or operators, laboratories, instruments, reagent lots, calibrators, etc.).

As used herein a progesterone metabolite is a compound derived from progesterone by biochemical mechanisms. A progesterone sulfate is a progesterone metabolite that has at least one sulfate group.

As used herein, the terms “purify” or “separate” or derivations thereof do not necessarily refer to the removal of all materials other than the analyte(s) of interest from a sample matrix. Instead, in some embodiments, the terms purify or separate refer to a procedure that enriches the amount of one or more analytes of interest relative to one or more other components present in the sample matrix. In some embodiments, a “purification” or “separation” procedure can be used to remove one or more components of a sample that could interfere with the detection of the analyte, for example, one or more components that could interfere with detection of an analyte by mass spectrometry.

As used herein, the term “selectivity” refers to the ability of the measurement procedure to accurately measure the analyte of interest without contribution of other substances potentially found within a sample. Selectivity may be expressed as cross-reactivity and/or response to substances other than analyte of interest in the presence of the analyte of interest.

As used herein, “selective reaction monitoring” or “SRM” is a technique in mass spectrometry whereby an ion of a particular mass is selected in the first stage of a tandem mass spectrometer and an ion product of a fragmentation reaction of the precursor ion is selected in the second stage of the mass spectrometer. In contrast, “multiple reaction monitoring” or “MRM” is the application of SRM to multiple product ions from one or more precursor ions. For example, in MRM where one or more different precursor ions are formed in the first stage, the ions may be selected sequentially and multiple product ions from detected. SRM monitors only a single fixed mass window, while MRM scans rapidly over multiple narrow mass windows to acquire traces of multiple fragment ion masses in parallel.

As used herein, the term “specificity” refers to the ability of the measurement procedure to discriminate the analyte of interest when presented with substances potentially found within a sample. Specificity may be expressed as percent cross-reactivity and/or response to substances other than analyte of interest in the absence of the analyte of interest.

The term “surface enhanced laser desorption ionization” or “SELDI” as used herein refers to a method in which a non-volatile sample is exposed to laser irradiation, which desorbs and ionizes analytes in the sample by various ionization pathways, including photo-ionization, protonation, deprotonation, and cluster decay. For SELDI, the sample is typically bound to a surface that preferentially retains one or more analytes of interest. As in MALDI, this process may also employ an energy-absorbing material to facilitate ionization.

As used herein, a “subject” may comprise an animal. Thus, in some embodiments, the sample is obtained from a mammalian animal, including, but not limited to a dog, a cat, a horse, a rat, a monkey, and the like. In some embodiments, the sample is obtained from a human subject. In some cases, the human subject is a pregnant female. In some embodiments, the subject is a patient, that is, a living person presenting themselves in a clinical setting for diagnosis, prognosis, or treatment of a disease or condition. In some embodiments, the sample is not a biological sample, but comprises a non-biological sample, e.g., obtained during the manufacture or laboratory analysis of a vitamin, which can be analyzed to determine the composition and/or yield of the manufacturing and/or analysis process.

As used herein, the term “ULOQ” or “Upper Limit of Quantitation” is the highest amount of analyte in a sample that can be quantitatively determined without dilution.

### Methods for Detecting Progesterone Sulfates

Embodiments of the present disclosure relate to methods and systems for the quantitative analysis of progesterone metabolite biomarkers. The present disclosure may be embodied in a variety of ways.

In one embodiment, disclosed is a method for determining the presence or amount of a progesterone metabolite in a sample from a subject by mass spectrometry comprising the steps of: (a) generating one or more precursor ions from the progesterone metabolite; (b) generating one or more product ions from the one or more precursor ions; (c) detecting the presence or amount of the one or more of the precursor ions generated in step (a) or the one or more product ions of step (b) or both; and relating the detected ions to the presence or amount of the progesterone metabolite in the sample. In certain embodiments, the method may comprise: providing a sample believed to contain at least one progesterone metabolite; optionally, chromatographically separating the at least one progesterone metabolite from other components in the sample; using mass spectrometry to generate one or more precursor ions and one more product ions from the one or more precursor ions that are specific to the progesterone metabolite; and determining the presence or amount of the progesterone metabolite in the sample.

In certain embodiments, the progesterone metabolite is a progesterone sulfate. In various embodiments, the progesterone metabolite may include at least one of 5β-Pregnan-3α, 20α-diol sulfate (PM3S); 5α-Pregnan-3α-ol-20-one sulfate (PM4S) and/or 5α-Pregnan-3β-ol-20-one sulfate (PM5S); and/or a diprogesterone sulfate such as 5α-Pregnan-3α, 20α-diol disulfate (PM2DiS) and/or 5β-Pregnan-3α, 20α-diol disulfate (PM3DiS). Additional nomenclature used interchangeably herein for the progesterone metabolites may include PM2DiS (allopregnanediol disulfate): 5α-Pregnan-3α, 20α-diol disulfate; PM3S (pregnanediol sulfate): 5β-Pregnan-3α, 20α-diol sulfate; PM3DiS (pregnanediol disulfate): 5β-Pregnan-3α, 20α-diol diSulfate; PM4S (allopregnanolone sulfate): 5α-Pregnan-3α-ol, 20-one sulfate; or PM5S (epiallopregnanolone sulfate): 5α-Pregnan-3β-ol, 20-one sulfate.

In an embodiment, the samples may be measured by tandem mass spectrometry (MS/MS). In certain embodiments, triple quadrupole tandem mass spectrometry may be used. In certain embodiments, multiple reaction monitoring (MRM), optionally with transition ion summing, may be used.

The analytes (i.e., progesterone metabolites of interest) may be partially purified prior to mass spectrometry. Thus, in an embodiment, the sample is subjected to a purification step prior to step (a) of generating a precursor ion. For example, in certain embodiments, the samples are subjected to dilution and/or precipitation of proteins. Or, liquid-liquid extraction (LLE) may be used. Or, solid-phase extraction (SPE) may be used.

In certain embodiments, the samples may be subjected to chromatography for purification. In an embodiment, the chromatography is liquid chromatography (LC) or high performance liquid chromatography (HPLC) or high throughput chromatography (HTLC). Thus, a variety of liquid chromatography separation techniques may be used. For example, the LC step may comprise one LC separation, or multiple LC separations. In one embodiment, the chromatographic separation comprises extraction and analytical liquid chromatography. As discussed herein, in certain embodiments, the analytical chromatography may comprise high performance liquid chromatography (HPLC). In certain embodiments, high turbulence liquid chromatography (HTLC) (also known as high throughput liquid chromatography) may be used. Additionally and/or alternatively, other types of chromatographic purification may be used.

Also, in certain embodiments, the methods of the present disclosure may comprise multiple liquid chromatography steps. Thus, in certain embodiments, a two-dimensional liquid chromatography (LC) procedure is used. For example, in one embodiment, the method may comprise transferring the biomarker of interest from the LC extraction column to an analytical column. In one embodiment, the transferring of the at least one biomarker of interest from the extraction column to an analytical column is done by a heart-cutting technique. In another embodiment, the biomarker of interest is transferred from the extraction column to an analytical column by a chromato-focusing technique. Alternatively, the biomarker of interest is transferred from the extraction column to an analytical column by a column switching technique. These transfer steps may be done manually, or may be part of an on-line system. Optionally, an extraction column may not be used in the methods and systems described herein.

Thus, in certain embodiments, the method may comprise the steps of: (a) providing a sample suspected of containing a progesterone metabolite; (b) partially purifying the progesterone metabolite from other components in the sample by sample dilution and/or protein precipitation; (c) transferring the progesterone metabolite to an analytical column and chromatographically separating the progesterone metabolite from other components in the sample; and (d) analyzing the chromatographically separated progesterone metabolite by mass spectrometry to determine the presence or amount of the one or more biomarkers in the test sample. In one embodiment, the mass spectrometry is tandem mass spectrometry.

In certain embodiments, five individual progesterone metabolites (e.g., PM3S, PM4S, PM5S, PM2DiS, and PM3DiS) may be measured by liquid chromatography with tandem mass spectrometry detection (LC-MS/MS) after dilution and protein precipitation.

In certain embodiments, certain of the progesterone metabolites may be measured together (i.e., simultaneously in the same assay) or separately (i.e., in different assays). For example, in certain embodiments, PM3S is measured in an assay individually (a “PM3S” assay). Also, in certain embodiments, the mono-sulfates PM4S and PM5S are measured in an assay together (a “MPMS” assay). Also in certain embodiments, the di-sulfates PM2DiS and PM3DiS are measured in an assay together (a “PMDiS” assay). In such embodiments, the three assays may use separate standards, quality control (QC) material, internal standards, and liquid chromatography tandem mass spectrometry (LC-MS/MS) methods. The steps in sample processing, however, may be the same for all three assays.

The method may include the use of internal standards. The internal standards may comprise the progesterone metabolite analyte of interest labeled with a stable isotope. In certain embodiments, the stable isotope may be deuterium (d). Or, other isotopes may be used. For example, in certain embodiments, the internal standard(s) may comprise at least one of: PM3S-d₄ (i.e., 5β-Pregnan-3α,20α-diol-[2,2,4,4-d4] sulfate); PM5S-d₄ (i.e., 5α-Pregnan-3β-ol-20-one-[2,2,4,4-d4] sulfate); PM2DiS-d₄ (i.e., 5α-Pregnan-3α,20α-diol-[2,2,4,4-d4] disulfate); and/or PM3DiS-d₄ (i.e., 5β-Pregnan-3α,20α-diol-[2,2,4,4-d4] disulfate). Such standards may be synthesized and/or purchased commercially.

Thus, in certain embodiments, progesterone sulfate stable isotope labeled internal standard (PM3S-d₄, PM5S-d₄, PM2DiS-d₄, and/or PM3DiS-d₄) may be added to standards, quality control, and patient samples (e.g., serum aliquots) to evaluate and correct for recovery of the individual progesterone sulfates from each sample. The standards, control samples, and patient samples are diluted and then undergo protein precipitation and/or other purifications steps. Then, a portion of the sample (i.e., sample, control and/or standards) may be concentrated by drying before reconstitution.

The final product from each patient and calibrator may then be analyzed by HPLC with tandem mass spectrometry. In certain embodiments, samples are injected onto the ARIA TX4 system where the analyte(s) of interest is chromatographed through an analytical column via a gradient separation. An AB SCIEX API5000 triple quadrupole mass spectrometer, operating in negative ion electrospray ionization (ESI) mode (Turboionspray) may be used for detection. Or, other types of ionization and/or methods of mass spectrometry as described herein may be used.

In certain embodiments, the back-calculated amount of analyte in each sample may be determined from duplicate calibration curves generated by spiking known amounts of purified progesterone sulfates into a matrix, e.g., 6% bovine serum albumin (BSA). Quantification of the analyte and internal standard may be performed in selected reaction monitoring mode (SRM) and/or multiple reaction monitoring (MRM). In certain embodiments, ion summing may be used.

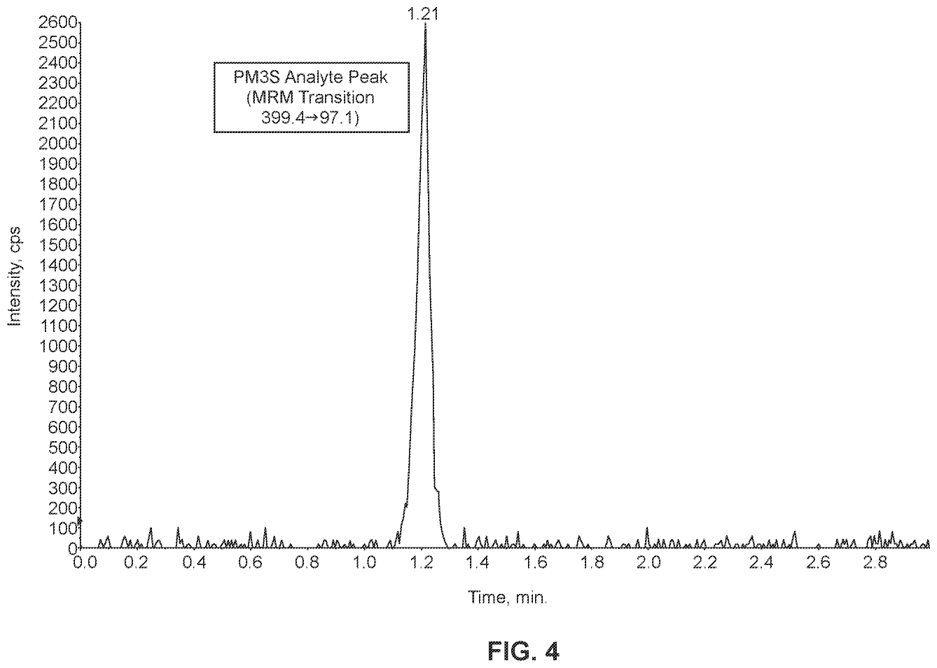

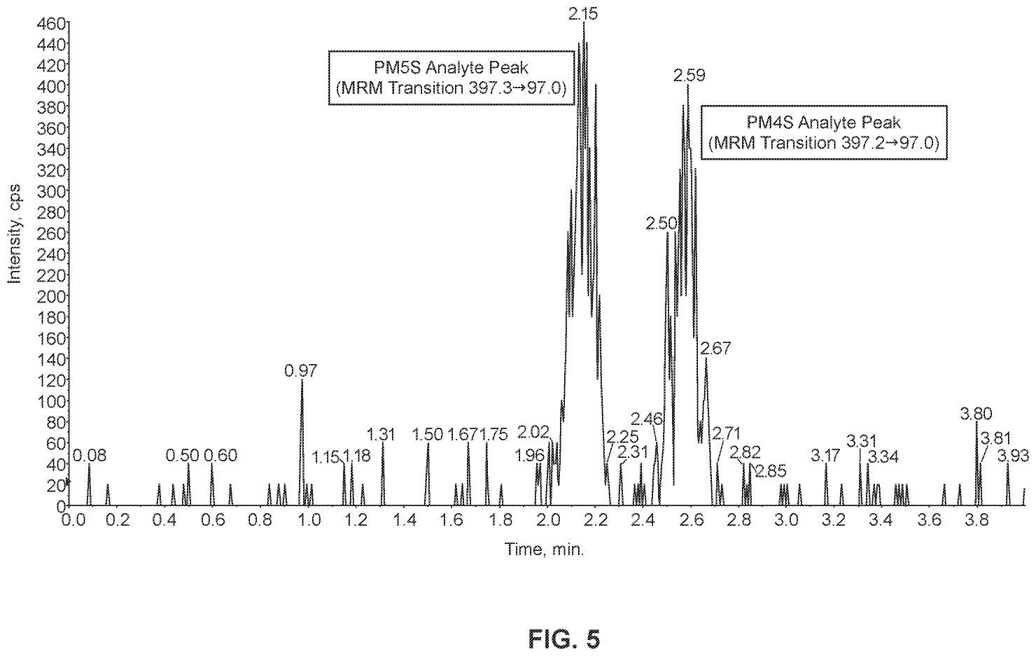

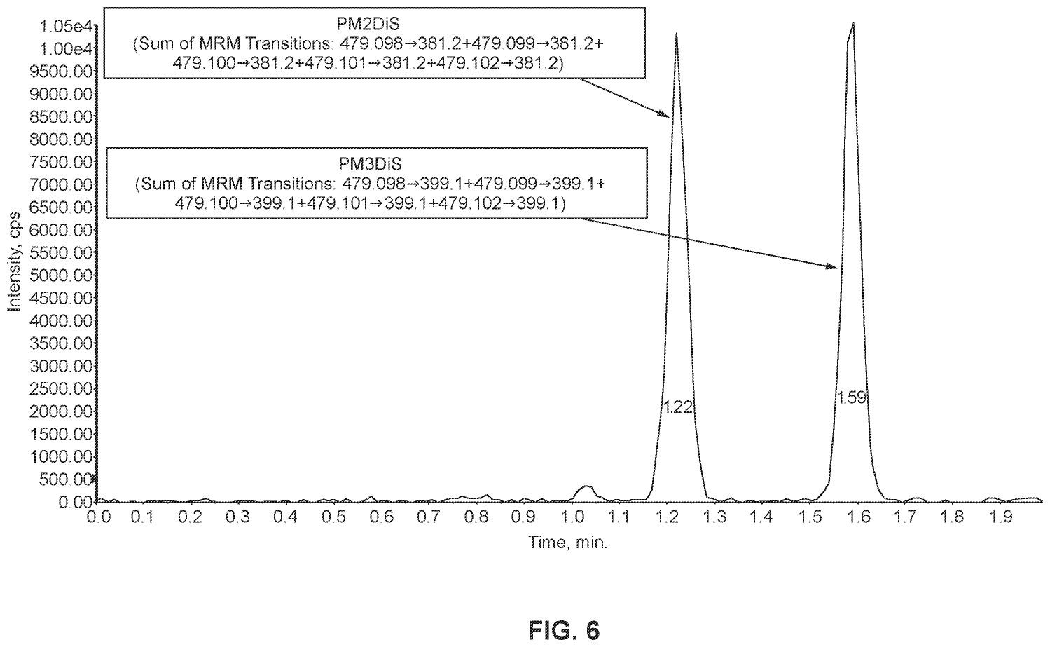

Example transitions that may be monitored are listed in Table 1. In an embodiment, LC-MS/MS acquisition may comprise the transitions as monitored in Tables 3-5. Or, other transitions may be monitored. As is known in the art, transitions may vary slightly from machine to machine and are determined during instrument tuning. For example, in certain embodiments, selected reaction monitoring (SRM) or multiple reaction monitoring (MRM) may be used to select parent (precursor) and product (i.e., daughter or fragment) ions. Examples of MRM scans for PM3S, MPMS (PM4S and PM5S), and PMDiS (PM2DiS and PM3DiS), selecting for the parent-daughter (i.e., precursor-product or precursor-fragment) ions are shown in FIGS. 4-6 respectively. Thus, in certain embodiments, and as shown in FIG. 4, PM3S is measured by MRM using the analyte peak for the transition of 399.400→97.100. Additionally and/or alternatively, in certain embodiments, and as shown in FIG. 5, PM4S and PM5S are measured by MRM using the analyte peak for the 397.3→97.0 transition for PM5S and the analyte peak for the 397.2→97.0 transition for PM4S. Additionally and/or alternatively, in certain embodiments, PM2DiS and PM3DiS are measured by MRM using ion summing. Thus, as shown in FIG. 6, the sum of 479.098→381.2+479.099→381.2+479.100→381.2+479.101→381.2+479.102→381.2 transitions may be used for detection of PM2DiS and the sum of 479.098→399.1+479.099→399.1+479.100→399.1+479.101→399.1+479.102→399.1 transitions may be used for detection of PM3DiS.

TABLE 1

| | | Analyte Ion | | Internal Standard | Ion |
|---|---|---|---|---|---|
| | | Transition | | Ion Transition | Summing at |
| | Analyte | (Parent→ | Internal Standard | (Parent→ | Quantitation |
| Assay | (Abbreviation) | Daugh\vec{te}r) | (Abbreviation) | Daugh\vec{te}r) | (Yes/No) |
| PM3S | 5β-Pregnan- | 399.4→97.1 | 5β-Pregnan- | 403.2→98.0 | No |
| | 3α,20α-diol sulfate | | 3α,20α-diol- | | |
| | (PM3S) | | [2,2,4,4-d4] sulfate | | |
| | | | (PM3S-IS) | | |
| Mono- | 5α-Pregnan-3α-ol- | 397.2→97.0 | 5α-Pregnan-3β-ol- | 401.2→97.8 | No |
| Progesterone | 20-one Sulfate | | 20-one-[2,2,4,4-d4] | | |
| Sulfates | (PM4S) | | Sulfate | | |
| (MPMS) | | | (PM5S-IS) | | |
| | 5α-Pregnan-3β-ol- | 397.3→97.0 | 5α-Pregnan-3β-ol- | 401.2→97.8 | No |
| | 20-one Sulfate | | 20-one-[2,2,4,4-d4] | | |
| | (PM5S) | | Sulfate (PM5S-IS) | | |
| Di- | 5α-Pregnan- | 479.1→381.2 | 5α-Pregnan- | 483.3→384.4 | Yes |
| Progesterone | 3α,20α-diol | | 3α,20α-diol- | | |
| Sulfates | Disulfate | | [2,2,4,4-d4] | | |
| (PMDiS) | (PM2DiS) | | Disulfate | | |
| | | | (PM2DiS-IS) | | |
| | 5β-Pregnan- | 479.1→399.1 | 5β-Pregnan- | 483.3→403 | Yes |
| | 3α,20α-diol | | 3α,20α-diol- | | |
| | Disulfate | | [2,2,4,4-d4] | | |
| | (PM3DiS) | | Disulfate | | |
| | | | (PM3DiS) | | |

A variety of samples may be used. In some embodiments, the biological sample may comprise blood, serum, plasma, urine, nasopharyngeal swabs, or saliva. In certain embodiments, the sample is serum. For example, serum may be collected in a red-top or SST tube and frozen. Plasma may be collected in a purple top (EDTA) or green-top (heparin) tube. In certain embodiments, a minimum of 1 mL serum, or 2.5 mL serum (e.g., for pediatric subjects) or 5 mL serum (adults) may be used.

In certain embodiments, using either samples of plasma or serum, the lower limit of detection (LLOD) for a sample aliquot of 100 μL is 1 ng/mL for each of PM3S, PM4S, PM5S, PM2DiS, and PM3DiS. Samples of a lower volume may be used. Thus, in certain embodiments a volume of 20 μL may be used for PM3S, PM2DiS, and PM3DiS and a volume of 10 μL may be used for PM4S and PM5S.

The method may comprise detection of a progesterone sulfate over an analytical measurement range (AMR) (i.e., LLOQ-ULOQ) from 1-500 ng/mL for PM3S, PM4S, PM5S PM2DiS and PM3DiS. PM3S, PM2DiS and PM3DiS can be diluted up to 5× for a maximum measurement of 2,500 ng/mL, while PM4S and PM5S can be diluted up to 10× for a maximum measurement of 5,000 ng/mL. In certain embodiments, the ULOQ is about 500 ng/mL and the LLOQ is 1 ng/mL for each of the progesterone sulfates.

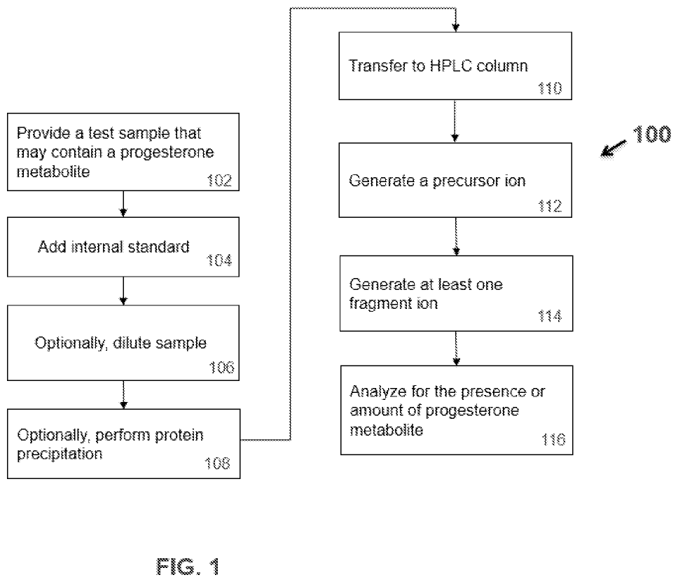

An example of a method of the present disclosure is shown in FIG. 1. Thus, in an embodiment, the method 100 may include a step of providing a biological sample, for example, a serum or plasma sample believed to contain a progesterone sulfate metabolite 102.

Next an internal standard may be added to the sample 104. In certain embodiments, progesterone sulfate stable isotope labeled internal standard (PM3S-d₄, PM5S-d₄, PM2DiS-d₄, and/or PM3DiS-d₄) are added to standards, quality control, and patient serum or plasma aliquots to evaluate and correct for recovery of the individual progesterone sulfates from each sample.

The sample (as well as needed standards and control samples) may then be diluted 106 and reagents to precipitate protein added 108. For example, in certain embodiments, samples are diluted by the addition of internal standard (IS) in 6% bovine serum albumin (BSA) and then acetonitrile is added to precipitate protein. A portion of the sample extract may then be concentrated by drying the sample before reconstitution.

Still referring to FIG. 1, the method may further include liquid chromatography as a means to separate the progesterone sulfate from other components in the sample. In certain embodiments, a single step of HPLC is used 110. Or, in some embodiments two liquid chromatography steps are used. For example, the method may comprise a first extraction column liquid chromatography (not shown) followed by transfer to a second analytical column (e.g., HPLC). Or, HTLC may be used.

A variety of analytical columns known in the art may be used as needed to provide good purification. In certain embodiments, the analytical column may comprise particles having an average diameter of about 2-3 μm (e.g., 2.6 μm). In some embodiments, the analytical column is a functionalized silica or polymer-silica hybrid, or a polymeric particle or monolithic silica stationary phase, such as a phenyl-hexyl functionalized analytical column. Or, in certain embodiments, an Aria TX4 HTLC System (Cohesive Technologies, MA) is used.

The separated analytes are then analyzed by mass spectrometry. In an embodiment, triple quadrupole tandem mass spectrometry (MS/MS) is used, whereby, one or more precursor ions are selected following ionization 112, and the one or more precursor ions are subjected to additional fragmentation to generate one or more product ions 114, whereby the one or more product ions are selected for detection.

The analyte of interest may then be quantified based upon the amount of the characteristic transitions measured by tandem MS 116. In some embodiments, the tandem mass spectrometer comprises a triple quadrupole mass spectrometer. In certain embodiments, an Applied Biosystems API5000 or API5500 in negative ESI mode may be used. In some embodiments, the tandem mass spectrometer is operated in a positive ion Atmospheric Pressure Chemical Ionization (APCI) mode. In some embodiments, the quantification of the analytes and internal standards is performed in the selected reaction monitoring mode (SRM). Or, other methods of ionization such as the use of inductively coupled plasma, or MALDI, or SELDI, or APPI may be used for ionization.

In some embodiments, the back-calculated amount of each analyte in each sample may be determined by comparison of unknown sample response or response ratio when employing internal standardization to calibration curves generated by spiking a known amount of purified analyte material into a standard test sample, e.g., charcoal stripped human serum or BSA. In one embodiment, calibrators are prepared at known concentrations and analyzed to generate a response or response ratio when employing internal standardization versus concentration calibration curve.

The disclosed methods provide the ability to quantify progesterone sulfates at physiologically relevant levels. As discussed herein, progesterone sulfate levels can be important indicators of the efficacy of various therapeutic compounds to reduce complications of pregnancy such as Intrahepatic Cholestasis of Pregnancy (ICP) and complications that can occur therefrom, such as prenatal death, preterm delivery, and/or iatrogenic preterm delivery. Thus, in some embodiments, the amount of the progesterone sulfate is used to distinguish whether gestational pruritus of the skin is an early symptom of (ICP) or due to benign pruritus gravidarum in the subject. In one embodiment, the method is able to report levels for PM3S, PM4S, PM5S, PM2DiS, and/or PM3DiS concentrations in serum as follows: PM3S: 36.0-221 ng/mL; and/or PM4S: 44.7-725 ng/mL; and/or PM5S: 11.2-223; PM2DiS: 37.4-505 ng/mL; and/or PM3DiS: 4.912-78.3 ng/mL.

### Systems for Analysis of Progesterone Sulfate Biomarkers

In other embodiments, the disclosure comprises a system for performing any of the steps of the methods disclosed herein. For example, in certain embodiments disclosed is a system for determining the presence or amount of one or more progesterone metabolites in a sample. In certain embodiments, the system for determining the presence or amount of a progesterone metabolite may comprise a station and/or component for providing a test sample suspected of contain a progesterone metabolite of interest; a mass spectrometer station and/or component for fragmentation of the progesterone metabolite of interest to generate at least one precursor ion and at least one product ion; and a station and/or component to determine the presence or amount of the progesterone metabolite of interest in the sample. In certain embodiments, the system may comprise a station and/or component for partially purifying the progesterone metabolite of interest from other components in the sample. Additionally and/or alternatively, the system may further comprise a station and/or component for chromatographically separating the progesterone metabolite of interest from other components in the sample. For example, the system may comprise: a station and/or component for providing a test sample; optionally, a station and/or component for partially purifying a progesterone metabolite from other components in the sample; optionally, a station and/or component for chromatographically separating the progesterone metabolite from other compounds in the sample; a station and/or component for mass spectrometry to generate one or more precursor ions and one or more product ions from the progesterone metabolite; and a station and/or component to analyze the mass spectrum to determine the presence or amount of the progesterone metabolite in the test sample. In some embodiments, certain of the stations and/or components are combined as single stations and/or components.

In certain embodiments, the progesterone metabolite is a progesterone sulfate. In various embodiments, the progesterone metabolite may include at least one of 5β-Pregnan-3α, 20α-diol sulfate (PM3S); 5α-Pregnan-3α-ol-20-one sulfate (PM4S) and/or 5α-Pregnan-3β-ol-20-one sulfate (PM5S); and/or a diprogesterone sulfate such as 5α-Pregnan-3α, 20α-diol disulfate (PM2DiS) and/or 5β-Pregnan-3α, 20α-diol disulfate (PM3DiS).

In an embodiment, the samples may be measured by tandem mass spectrometry. In certain embodiments, triple quadrupole tandem mass spectrometry (MS/MS) may be used. In certain embodiments, selective reaction monitoring (SRM) or multiple reaction monitoring (MRM), optionally with transition ion summing, may be used.

In certain embodiments, at least one of the stations may be controlled by a computer and/or a computer-program product tangibly embodied in a non-transitory machine-readable storage medium. Thus, in certain embodiments the system may comprise a computer-program product tangibly embodied in a non-transitory machine-readable storage medium, including instructions configured to control any of the stations and/or components of the system.

The system may include a station and/or component for the addition of a stable isotope-labeled internal standard(s). In certain embodiments, a progesterone sulfate stable isotope labeled internal standard (e.g., PM3S-d₄, PM5S-d₄, PM2DiS-d₄, and/or PM3DiS-d₄) is added to standards, quality control, and patient serum or plasma aliquots to evaluate and correct for recovery of the individual progesterone sulfates from each sample.

In certain embodiments, the system may also comprise a station and/or component for partially purifying the at least one progesterone metabolite of interest from other components in the sample prior to the station for liquid chromatographic separation. In certain embodiments, the station and/or component for partial purification may comprise a station or component for dilution of the sample, liquid-liquid extraction, solid phase extraction and/or precipitation of proteins. For example, the station and/or component for partial purification may comprise reagents for dilution and/or protein precipitation.

In certain embodiments, system may comprise a station and/or component for chromatographically separating the progesterone metabolite from other components in the sample. In various embodiments, the chromatography is liquid chromatography (LC) or high performance liquid chromatography (HPLC) or high throughput chromatography (HTLC). The LC step may comprise one LC separation, or multiple LC separations. In one embodiment, the chromatographic separation comprises extraction and analytical liquid chromatography. As discussed herein, in certain embodiments, the analytical chromatography may comprise high performance liquid chromatography (HPLC). In certain embodiments, high turbulence liquid chromatography (HTLC) (also known as high throughput liquid chromatography) may be used. Additionally and/or alternatively, other types of purification may be used.

As noted herein, in some embodiments an isotopically-labeled internal standard or standards may be added to the sample. The isotopically-labeled internal standard or standards may be added prior to the partial purification step to standardize losses of the analyte (e.g., a progesterone metabolite) that may occur during the procedures. Thus, the station and/or component for partial purification may comprise a hood or other safety features required for working with solvents and/or isotope-labeled materials.

The stations or components for sample dilution, protein precipitation and addition of internal standards may each be individual stations, or they may be combined as one or two stations or components.

In one embodiment, the station and/or component for chromatographic separation comprises at least one apparatus to perform liquid chromatography (LC). In one embodiment, the station and/or component for liquid chromatography may comprise a column for analytical chromatography. For example, in certain embodiments, the chromatography may comprise high performance liquid chromatography (HPLC). Or, in some embodiments, the chromatography may comprise HTLC. In some embodiments, the chromatographic separation may also comprises extraction chromatography prior to the analytical liquid chromatography.

A variety of analytical columns known in the art may be used as part of the chromatographic station as needed to provide good purification. In certain embodiments, the analytical column may comprise particles having an average diameter of about 2-3 μm (i.e., 2.6 μm). In some embodiments, the analytical column is a functionalized silica or polymer-silica hybrid, or a polymeric particle or monolithic silica stationary phase, such as a phenyl-hexyl functionalized analytical column. Or, in certain embodiments, the station may comprise an Aria TX4 HTLC System (Cohesive Technologies, MA).

In certain embodiments, the systems of the present disclosure may comprise multiple liquid chromatography steps. Thus, in certain embodiments, a two-dimensional liquid chromatography (LC) procedure is used. For example, in one embodiment, systems of the present disclosure may comprise a station and/or component for transferring the biomarker of interest from the LC extraction column to an analytical column. In one embodiment, the transferring of the at least one biomarker of interest from the extraction column to an analytical column is done by a heart-cutting technique. In another embodiment, the biomarker of interest is transferred from the extraction column to an analytical column by a chromato-focusing technique. Alternatively, the biomarker of interest is transferred from the extraction column to an analytical column by a column-switching technique. These transfer steps may be done manually, or may be part of an on-line system. Optionally, an extraction column may not be used in the methods and systems described herein.

In certain embodiments, the station and/or component for mass spectrometry comprises a tandem mass spectrometer. In some embodiments, the tandem mass spectrometer comprises a triple quadrupole mass spectrometer. In certain embodiments, an Applied Biosystems API5000, API5500 or an Agilent 7000 triple quadrupole mass spectrometer in negative ESI mode may be used. In some embodiments, the tandem mass spectrometer is operated in a positive ion Atmospheric Pressure Chemical Ionization (APCI) mode. In some embodiments, the quantification of the analytes and internal standards is performed in the selected reaction monitoring mode (SRM). Or, other methods of ionization such as the use of inductively coupled plasma, or MALDI, or SELDI, or APPI may be used for ionization.

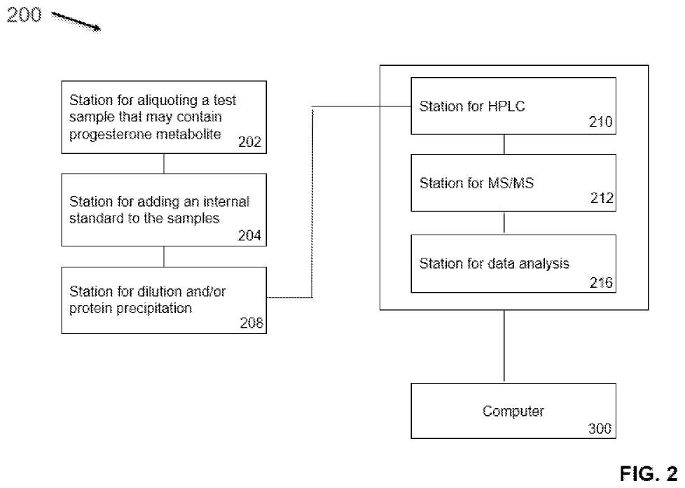

FIG. 2 provides a drawing of an embodiment of a system of the disclosure. As shown in FIG. 2, the system 200 may comprise a station and/or component for aliquoting a sample that may comprise a progesterone metabolite of interest into sampling containers 202. In one embodiment, the sample is aliquoted into a container or containers to facilitate liquid-liquid extraction or sample dilution. The station and/or component for aliquoting may comprise receptacles to discard or store the portion of the biological sample that is not used in the analysis.

The system may further comprise a station and/or component for adding an internal standard to the sample 204. In an embodiment, the internal standard comprises at least one of the progesterone metabolites of interest labeled with a non-natural isotope. Thus, the station or component for adding an internal standard may comprise safety features to facilitate adding an isotopically labeled internal standard solutions to the sample.

The system may also, in some embodiments, comprise a station(s) and/or component(s) for partial purification of the progesterone metabolite of interest 208. Thus, in certain embodiments, the system may comprise a station or component for dilution of the sample and/or protein precipitation 208. Or, stations and/or components for other types of sample purification, such as liquid-liquid extraction or solid phase extraction may be included.

The system may also comprise a station and/or component for liquid chromatography (LC) of the sample 210. As described herein, in an embodiment, the station and/or component for liquid chromatography may comprise an HPLC (or HTLC) column. The station for liquid chromatography may comprise a column comprising the stationary phase, as well as containers or receptacles comprising solvents that are used as the mobile phase. In an embodiment, the mobile phase comprises a gradient of acetonitrile, ammonium formate, and water, or other miscible solvents with aqueous volatile buffer solutions. Thus, in one embodiment, the station and/or component for chromatography may comprise the appropriate lines and valves to adjust the amounts of individual solvents being applied to the column or columns. Also, the station and/or component may comprise a means to remove and discard those fractions that do not comprise the biomarker of interest. In an embodiment, the fractions that do not contain the biomarker of interest are continuously removed from the column and sent to a waste receptacle for decontamination and to be discarded. In certain embodiments, the station and/or component may comprise an Aria TX4 HTLC System (Cohesive Technologies, MA).

Also, the system may comprise a station and/or component for mass spectrometry 212. In an embodiment, the station or component for mass spectrometry comprises tandem mass spectrometry (MS/MS). Also, the system may comprise a station and/or component for characterization and/or quantification of the analyte. The station and/or component for characterization and/or quantification may comprise a station and/or component for data analysis 216 of the LC-MS/MS results. In an embodiment, the analysis comprises both identification and quantification of the progesterone metabolite(s) of interest.

As illustrated in FIG. 2, any of the stations and/or components of the system may be automated, robotically controlled, and/or controlled at least in part by a computer 300 and/or programmable software. For example, the station(s) and/or components(s) for LC-MS/MS and/or data analysis may be controlled at least in part, by a computer. Thus, the system may comprise a computer-program product tangibly embodied in a non-transitory machine-readable storage medium, including instructions configured to run the system or any part (e.g., station or component) of the system and/or perform a step or steps of the methods of any of the disclosed embodiments. In some embodiments, a system is provided that includes one or more data processors and a non-transitory computer readable storage medium containing instructions which, when executed on the one or more data processors, cause the one or more data processors to perform part or all of one or more methods or processes disclosed herein and/or run any of the parts of the systems disclosed herein.

For example, disclosed is a system comprising one or more data processors, and a non-transitory computer readable storage medium containing instructions which, when executed on the one or more data processors, cause the one or more data processors to perform actions to direct at least one of the steps of: providing a sample believed to contain at least one progesterone metabolite; optionally, chromatographically separating the at least one progesterone metabolite from other components in the sample; using tandem mass spectrometry to generate at least one precursor ion and at least one product ion specific to the progesterone metabolite; and determining the presence or amount of the progesterone metabolite in the sample. Additionally and/or alternatively, in certain embodiments disclosed is a system comprising one or more data processors, and a non-transitory computer readable storage medium containing instructions which, when executed on the one or more data processors, cause the one or more data processors to perform actions to direct at least one of the steps of: (a) generating one or more precursor ions from the progesterone metabolite; (b) generating one or more product ions of the precursor ion; (c) detecting the presence or amount of the one or more of the precursor ion generated in step (a) or the one or more product ions of step (b) or both; and relating the detected ions to the presence or amount of the progesterone metabolite in the sample.

Also disclosed is a computer-program product tangibly embodied in a non-transitory machine-readable storage medium, including instructions configured to run any of the components or stations of the disclosed systems and/or perform a step or steps of the methods of any of the disclosed embodiments. For example, in certain embodiments, the computer-program product tangibly embodied in a non-transitory machine-readable storage medium includes instructions configured to cause one or more data processors to perform actions to direct at least one of the steps of providing a sample believed to contain at least one progesterone metabolite; optionally, chromatographically separating the at least one progesterone metabolite from other components in the sample; using tandem mass spectrometry to generate at least one precursor ion and at least one product ion specific to the progesterone metabolite; and determining the presence or amount of the progesterone metabolite in the sample. In certain embodiments disclosed is a non-transitory computer readable storage medium containing instructions which, when executed on the one or more data processors, cause the one or more data processors to perform actions to direct at least one of the steps of: (a) generating a precursor ions from the progesterone metabolite; (b) generating one or more product ions of the precursor ion; (c) detecting the presence or amount of one or more of the precursor ion generated in step (a) or the one or more product ions of step (b) or both; and relating the detected ions to the presence or amount of the progesterone metabolite in the sample.

The systems and computer products may perform any of the methods disclosed herein. One or more embodiments described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines.

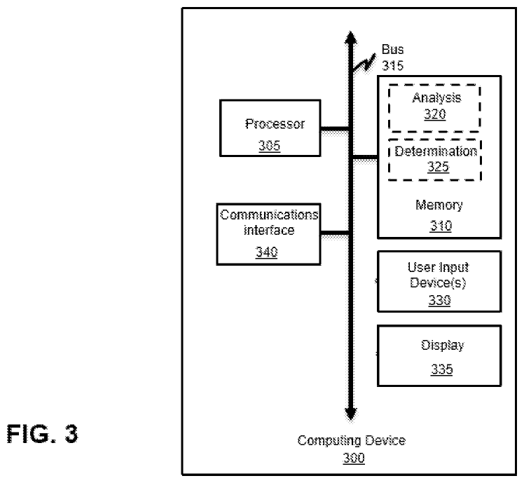

FIG. 3 shows a block diagram of an analysis system 300 used for detection and/or quantification of a progesterone metabolite. As illustrated in FIG. 3, modules, engines, or components (e.g., program, code, or instructions) executable by one or more processors may be used to implement the various subsystems of an analyzer system according to various embodiments. The modules, engines, or components may be stored on a non-transitory computer medium. As needed, one or more of the modules, engines, or components may be loaded into system memory (e.g., RAM) and executed by one or more processors of the analyzer system. In the example depicted in FIG. 3, modules, engines, or components are shown for implementing the methods or running any of the systems of the disclosure.

Thus, FIG. 3 illustrates an example computing device 300 suitable for use with systems and the methods according to this disclosure. The example computing device 300 includes a processor 305 which is in communication with the memory 310 and other components of the computing device 300 using one or more communications buses 315. The processor 305 is configured to execute processor-executable instructions stored in the memory 310 to perform one or more methods or operate one or more stations for detecting progesterone metabolite levels according to different examples, such as those in FIGS. 1-2 and 4-6 or disclosed elsewhere herein. In this example, the memory 310 may store processor-executable instructions 325 that can analyze 320 results for sample as discussed herein.

The computing device 300 in this example may also include one or more user input devices 330, such as a keyboard, mouse, touchscreen, microphone, etc., to accept user input. The computing device 300 may also include a display 335 to provide visual output to a user such as a user interface. The computing device 300 may also include a communications interface 340. In some examples, the communications interface 340 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

In some embodiments, one or more of the purification or separation steps can be performed “on-line.” The on-line system may comprise an autosampler for removing aliquots of the sample from one container and transferring such aliquots into another container. For example, an autosampler may be used to transfer the sample after extraction onto an LC extraction column. Additionally or alternatively, the on-line system may comprise one or more injection ports for injecting the fractions isolated from the LC extraction columns onto the LC analytical column. Additionally or alternatively, the on-line system may comprise one or more injection ports for injecting the LC purified sample into the MS system. Thus, the on-line system may comprise one or more columns, including but not limited to, an extraction column, and/or an analytical column. Additionally or alternatively, the system may comprise a detection system, e.g., a mass spectrometer system. The on-line system may also comprise one or more pumps; one or more valves; and necessary plumbing. In such “on-line” systems, the test sample and/or analytes of interest can be passed from one component of the system to another without exiting the system, e.g., without having to be collected and then disposed into another component of the system.

In some embodiments, the on-line purification or separation method can be automated. In such embodiments, the steps can be performed without the need for operator intervention once the process is set-up and initiated. For example, in one embodiment, the system, or portions of the system may be controlled by a computer or computers 300. Thus, in certain embodiments, the present disclosure may comprise software for controlling the various components of the system, including pumps, valves, autosamplers, and the like. Such software can be used to optimize the extraction process through the precise timing of sample and solute additions and flow rate.

Although some or all of the steps in the method and the stations comprising the system may be on-line, in certain embodiments, some or all of the steps may be performed “off-line.” In such off-line procedures, the analytes of interests typically are separated, for example, on an extraction column or by liquid/liquid extraction, from the other components in the sample matrix and then collected for subsequent introduction into another chromatographic or detector system. Off-line procedures typically require manual intervention on the part of the operator.

As noted herein, liquid chromatography may, in certain embodiments, comprise high turbulence liquid chromatography or high throughput liquid chromatography (HTLC). See, e.g., Zimmer et al., J. Chromatogr. A 854:23-35 (1999); see also, U.S. Pat. Nos. 5,968,367; 5,919,368; 5,795,469; and 5,772,874. Traditional HPLC analysis relies on column packing in which laminar flow of the sample through the column is the basis for separation of the analyte of interest from the sample. In such columns, separation is a diffusional process. Turbulent flow, such as that provided by HTLC columns and methods, may enhance the rate of mass transfer, improving the separation characteristics provided. In some embodiments, high turbulence liquid chromatography (HTLC), alone or in combination with one or more purification methods, may be used to purify the biomarker of interest prior to mass spectrometry. In such embodiments, samples may be extracted using an HTLC extraction cartridge which captures the analyte, then eluted and chromatographed on a second HTLC column or onto an analytical HPLC column prior to ionization. Because the steps involved in these chromatography procedures can be linked in an automated fashion, the requirement for operator involvement during the purification of the analyte can be minimized. Also, in some embodiments, the use of a high turbulence liquid chromatography sample preparation method can eliminate the need for other sample preparation methods including liquid-liquid extraction. Thus, in some embodiments, the test sample, e.g., a biological fluid, can be disposed, e.g., injected, directly onto a high turbulence liquid chromatography system.

For example, in a typical high turbulence or turbulent liquid chromatography system, the sample may be injected directly onto a narrow (e.g., 0.5 mm to 2 mm internal diameter by 20 to 50 mm long) column packed with large (e.g., >25 micron) particles. When a flow rate (e.g., 3-500 mL per minute) is applied to the column, the relatively narrow width of the column causes an increase in the velocity of the mobile phase. The large particles present in the column can prevent the increased velocity from causing back pressure and promote the formation of vacillating eddies between the particles, thereby creating turbulence within the column.

In high turbulence liquid chromatography, the analyte molecules may bind quickly to the particles and typically do not spread out, or diffuse, along the length of the column. This lessened longitudinal diffusion typically provides better, and more rapid, separation of the analytes of interest from the sample matrix. Further, the turbulence within the column reduces the friction on molecules that typically occurs as they travel past the particles. For example, in traditional HPLC, the molecules traveling closest to the particle move along the column more slowly than those flowing through the center of the path between the particles. This difference in flow rate causes the analyte molecules to spread out along the length of the column. When turbulence is introduced into a column, the friction on the molecules from the particle is negligible, reducing longitudinal diffusion.

In certain embodiments, the mass spectrometer uses a “quadrupole” system. In a “quadrupole” or “quadrupole ion trap” mass spectrometer, ions in an oscillating radio frequency (RF) field experience a force proportional to the direct current (DC) potential applied between electrodes, the amplitude of the RF signal, and m/z. The voltage and amplitude can be selected so that only ions having a particular m/z travel the length of the quadrupole, while all other ions are deflected. Thus, quadrupole instruments can act as both a “mass filter” and as a “mass detector” for the ions injected into the instrument.

In certain embodiments, tandem mass spectrometry, or “MS/MS” is used. MS/MS methods are useful for the analysis of complex mixtures, especially biological samples, in part because the selectivity of MS/MS can minimize the need for extensive sample clean-up prior to analysis.

In an embodiment, the methods and systems of the present disclosure use a triple quadrupole MS/MS. Triple quadrupole MS/MS instruments typically consist of two quadrupole mass filters separated by a fragmentation means. In one embodiment, the instrument may comprise a quadrupole mass filter operated in the RF only mode as an ion containment or transmission device. In an embodiment, the quadrupole may further comprise a collision gas at a pressure of between 1 and 10 millitorr. Many other types of “hybrid” tandem mass spectrometers are also known, and can be used in the methods and systems of the present disclosure including various combinations of magnetic sector analyzers and quadrupole filters. These hybrid instruments often comprise high resolution magnetic sector analyzers (i.e., analyzers comprising both magnetic and electrostatic sectors arranged in a double-focusing combination) as either or both of the mass filters. Use of high resolution mass filters may be highly effective in reducing chemical noise to very low levels.

For the methods and systems of the present disclosure, ions can be produced using a variety of methods including, but not limited to, electron ionization, chemical ionization, fast atom bombardment, field desorption, and matrix-assisted laser desorption ionization (“MALDI”), surface enhanced laser desorption ionization (“SELDI”), photon ionization, electrospray ionization, and inductively coupled plasma.

Embodiments of the present disclosure may provide certain advantages. In certain embodiments, the methods and systems of the present disclosure may provide greater sensitivity than the sensitivities previously attainable for many of the analytes being measured.

Also, embodiments of the methods and systems of the present disclosure may provide for rapid throughput that has previously not been attainable for many of the analytes being measured. For example, using the methods and systems of the present disclosure, multiple samples may be analyzed for progesterone sulfate analytes using 96 well plates and a multiplex system of four LC-MS/MS systems, significantly increasing the throughput.

As another advantage, the specificity and sensitivity provided by the methods and systems of the present disclosure may allow for the analysis of analytes from a variety of biological materials. For example, the LC-MS/MS methods of the present disclosure can be applied to the quantification of analytes of interest in complex sample biological matrices, including, but not limited to, blood, serum, plasma, urine, saliva, nasopharyngeal swabs and the like. Thus, the methods and systems of the present disclosure are suitable for clinical applications and/or clinical trials.

As additional potential advantages, in certain embodiments, the systems and methods of the present disclosure provide approaches for addressing isobaric interferences, varied sample content, including hemolyzed and lipemic samples, while attaining low mg/dL limits of quantification (LOQ) of the target analytes. Accordingly, embodiments of the methods and systems of the present disclosure may provide for the quantitative, sensitive, and specific detection of clinical biomarkers used in the clinical diagnosis of disorders.

### EXAMPLES

The disclosure may be better understood by reference to the following non-limiting examples.

### Example 1—Assay Method

Five individual Progesterone Sulfates (PM3S, PM4S, PM5S, PM2DiS, and PM3DiS) were measured by liquid chromatography with tandem mass spectrometry detection (LC-MS/MS) after dilution and protein precipitation. PM3S was measured in an assay individually (PM3S). PM4S and PM5S were measured in an assay together (MPMS) while PM2DiS and PM3DiS were measured in an assay together (PMDiS). The three assays use separate standards, QC material, Internal Standard, and LCMS methods. The steps in sample processing are the same for all three assays.

Progesterone sulfate stable isotope labeled internal standards (PM3S-d₄, PM5S-d₄, PM2DiS-d₄, and/or PM3DiS-d₄) were added to standards, quality control, and patient serum aliquots to evaluate and correct for recovery of the individual progesterone sulfates from each sample. The standards, control samples, and patient serum were diluted and then underwent protein precipitation. A portion of the sample extract was concentrated by drying the sample before reconstitution. The final product from each patient and calibrator was analyzed by HPLC with tandem mass spectrometry. All samples were injected onto an ARIA TX4 system where the analyte(s) of interest were chromatographed through an analytical column via a gradient separation. An AB SCIEX API5000 triple quadrupole mass spectrometer, operating in negative ion electrospray ionization (ESI) mode (Turboionspray) was used for detection.

The back-calculated amount of analyte in each sample was determined from duplicate calibration curves generated by spiking known amounts of purified Progesterone Sulfates into 6% BSA. Quantification of analyte and internal standard was performed in selected reaction monitoring (SRM) mode with the use of ion summing when necessary. The transitions monitored are listed in the table below. Note that transitions may vary slightly from machine to machine and are determined during instrument tuning.

### Example 2—Validation of LC-MS/MS Assays for Progesterone Sulfates

Chromatographic Assays—Validation runs include a minimum of quality controls (QC) at three concentration levels (low, medium, high).

Calibration acceptance criteria—Calibration curves have a minimum of 6 non-zero concentration levels and a blank. Read back concentrations must be within 15% of nominal concentration (20% at LLOQ) in at least 75% of the levels.

QC acceptance criteria—At least ⅔ of QC are ±15% of target values; at least 50% of QC at each concentration level are ±15% of target values.

Short term storage, long term storage and freeze thaw stability—Serum stored at the following conditions is acceptable to use for the analysis of PM3S, PM4S, PM5S, PM2DiS, and PM3DiS concentration: Frozen (≤−10° C. and ≤−55° C.): 27 days; Refrigerated (2-8° C.): 14 days; Room temperature (15-30° C.): 14 days; or Freeze/thaw cycles (≤−10° C.): 6 cycles (7 total thaws). Long term storage for serum frozen (≤−10° C.) is 27 days and samples may be shipped frozen on dry ice.

Reference Intervals

To establish a reference interval, one hundred and twenty pregnant female human serum samples were analyzed and all samples were used for reference interval evaluation. The individual serum (SST) samples were collected from women in the 15-24 week range of pregnancy for AFP Tetra analysis. The reference intervals for PM3S, PM4S, PM5S, PM2DiS, and PM3DiS concentrations in serum as determined by the 97.5^{th} percentile are listed below:
PM3S: 36.0-221 ng/mL
PM4S: 44.7-725 ng/mL
PM5S: 11.2-223 ng/mL
PM2DiS: 37.4-505 ng/mL
PM3DiS: 4.912-78.3 ng/mL

Example scans are shown in FIGS. 4-6. In these experiments multiple reaction monitoring was used to select the appropriate transitions. PM3S was monitored in an assay individually (PM3S) (FIG. 4). PM4S and PM5S were monitored in an assay together (FIG. 5). PM2DiS and PM3DiS were monitored in an assay together (FIG. 6). Ion summing was used for the analysis and measurement of PM2DiS and PM3DiS).

Results are summarized in Table 2 where analytes that pass the acceptance criteria are shown in the Analytes column.

TABLE 2

| | | ACCEPTANCE | |
|---|---|---|---|
| PARAMETER | MATERIAL | CRITERIA | ANALYTES |
| Intra-assay | Five levels of each progesterone | Runs include an LLOQ | PM3S, PM4S, |
| Standard | sulfate were diluted in 6% BSA at | calibrator level | PM5S, PM2DiS, |
| Accuracy and | LLOQ, low, mid, high, and ULOQ | Exempt from QC acceptance | and PM3DiS |
| Precision | target concentrations. Preparation | criteria | |
| | of accuracy samples was | Bias ≤± 15%; (LLOQ ± | |
| | independent of standard | 20%) | |
| | preparation. Twenty replicates of | CV ≤ 15%; (LLOQ ≤ 20%) | |
| | each level was analyzed in a single | | |
| | batch. | | |
| Inter-assay | The above samples were analyzed | Runs include an LLOQ | PM3S, PM4S, |
| Standard | in an additional 5 batches, testing | calibrator level | PM5S, PM2DiS, |
| Accuracy and | six replicates at each level, using | Exempt from QC acceptance | and PM3DiS |
| Precision | different reagent lots as available. | criteria | |
| | | Mean inter-assay bias | |
| | | ≤± 15%; (LLOQ ± 20%) and at | |
| | | least ⅔ of intra-assay bias | |
| | | values ≤± 15% | |
| | | CV ≤ 15%; (LLOQ ≤ 20%) | |
| | | and at least ⅔ of intra-assay | |
| | | CV values within range | |
| Intra-assay | Human serums with high, | CV ≤ 15%; (LLOQ ≤ 20%) | PM3S, PM4S, |
| Sample Precision | medium, and low concentrations | | PM5S, PM2DiS, |
| | of each progesterone sulfate | | and PM3DiS |
| | (spiked when necessary) were | | |
| | used for analysis. Each | | |
| | concentration was analyzed six | | |
| | times within one assay batch. | | |
| Inter-assay | The above samples were analyzed | CV ≤ 15%; (LLOQ ≤ 20%) | PM3S, PM4S, |
| Sample Precision | in three separate assay batches. | and at least ⅔ of intra-assay | PM5S, PM2DiS, |
| | Each sample was analyzed six | CV values within range | and PM3DiS |
| | times within a single assay batch. | | |
| Lower Limit of | Progesterone sulfate was diluted in | Lowest concentration | PM3S, PM4S, |
| Quantitation | 6% BSA. Inaccuracy and | meeting accuracy and precision | PM5S, PM2DiS, |
| (LLOQ) | Imprecision data was used. | criteria | and PM3DiS |
| | | Response at LLOQ is ≥5 | 1 ng/mL |
| | | times the response of zero | |
| | | calibrator | |
| Upper Limit of | Progesterone sulfate was diluted in | Highest concentration meeting | PM3S, PM4S, |
| Quantitation | 6% BSA. Inaccuracy and | accuracy and precision criteria | PM5S, PM2DiS, |
| (ULOQ) | Imprecision data was used. | | and PM3DiS |
| | | | 500 ng/mL |
| Blank Matrix | Six lots of blank matrix (6% BSA) | Blank and zero calibrator are | PM3S, PM4S, |
| Effect | was analyzed in triplicate | free of interference at the | PM5S, PM2DiS, |
| | | retention times of analyte and | and PM3DiS |
| | | Internal Standard | |
| | | Response of lowest standard | |
| | | is at least 5 times blank response | |
| | | Double blank response at the | |
| | | IS retention time is ≤5% of | |
| | | average IS of calibrators and QC | |
| | | in the same run | |
| Internal Standard | Internal Standard in blank matrix | Blank with IS added <LLOQ. | PM3S, PM4S, |
| Interference | was injected as sample and | | PM5S, PM2DiS, |
| | analyzed in triplicate in a single | | and PM3DiS |
| | batch. | | |
| Effect of | Icteric, lipemic, and hemolyzed | Recovery from baseline is | PM3S, PM4S, |
| Lipemic, Icteric, | samples were prepared by adding | 85-115% in at least ⅔ of the | PM5S, PM2DiS, |
| and Hemolyzed | using the Sun Assurance ® | samples tested at each condition. | and PM3DiS |
| samples | Interference Kit as directed by the | | for icteric, lipemic, |
| | manufacturer. Three replicates | | and hemolyzed |
| | were tested for each sample type | | samples. |
| | and baseline. | | |
| Collection Tube | Red top serum, SST serum, and | Recovery from red top serum | PM3S, PM4S, |
| Type | plasma collection tubes from three | collection is 85-115% in at least | PM5S, PM2DiS, |
| | donors were analyzed and | ⅔ of the samples tested for | and PM3DiS |
| | compared. Three replicates from | each alternative tube type. | |
| | each donor tube type were | | |
| | analyzed. | | |
| LC system carry- | A blank following a high sample | Response of the blank | PM3S, PM4S, |
| over evaluation | was analyzed in at least three runs | following a high sample should | PM5S, PM2DiS, |
| | for each Progesterone Sulfate. | be less than the LLOQ. | and PM3DiS |
| | | maximum of 3SD of the low | |
| | | sample. | |
| Spike and | Human serum and calibrator with | Mean % Recovery from | PM3S, PM4S, |
| Recovery | low level concentrations of | expected (baseline concentration | PM5S, PM2DiS, |
| | progesterone sulfate were spiked | plus spike) 85-115% (80-120% | and PM3DiS |
| | with the Progesterone Sulfate | at LLOQ) | |
| | standard materials to low, mid and | At least two-thirds of the | |
| | high concentrations. Baseline and | sample replicates tested within | |
| | spiked samples were tested in | 85-115% recovery. | |
| | triplicate. | | |
| Dilution | Reduced volume and then diluted | CV ≤ 15%; (LLOQ ≤ 20%) | PM3S, PM2DiS, |
| Linearity (AMR | serum was analyzed. Three | 85-115% of expected values | and PM3DiS: |
| verification) | individual donors were analyzed at | (based on measurement of neat, | X2 and X5 dilution |
| | each dilution level. Five replicates | undiluted serum or plasma) at | PM4S and PM5S: |
| | per concentration level were | each dilution level (80-120% if | X2, X5, and X10 |
| | analyzed. | near LLOQ) | dilution |
| Autosampler | Autosampler stability was | Mean post-storage recovery 85- | PM3S, PM4S, |
| Stability | evaluated using calibrators and | 115% (80-120% at LLOQ) of | PM5S, PM2DiS, |
| | quality control samples. Duplicate | the mean pre-storage | and PM3DiS |
| | sample sets were included with the | concentration with at least two- | |
| | batch. The first sample set was | thirds of the sample replicates | |
| | injected, then after a minimum of | tested within 85-115% recovery. | |
| | 48 hours the entire batch was | | |
| | injected or re-injected. | | |
| Short-term | Short-term sample stability was | Mean % Recovery from | PM3S, PM4S, |
| Stability | determined by testing freshly | baseline 85-115% (80-120% at | PM5S, PM2DiS, |
| | collected human serum that was | LLOQ), with at least two-thirds | and PM3DiS |
| | spiked and then stored under | of the sample replicates at a | 27 days frozen |
| | conditions likely to be encountered | particular condition tested within | (≤−10° C. and ≤− |
| | in sample handling and laboratory | 85-115% recovery. | 55° C.) |
| | analysis. | | 14 days |
| | | | refrigerated (2-8° C.) |
| | | | and room |
| | | | temperature (15- |
| | | | 30° C.) |
| Freeze/thaw | Sample freeze/thaw stability was | Mean % Recovery from | PM3S, PM4S, |
| Stability | determined using aliquots of the | baseline 85-115% (80-120% at | PM5S, PM2DiS, |
| | collected spiked human serum | LLOQ), with at least two-thirds | and PM3DiS |
| | used to evaluate short-term | of the sample replicates at a | 6 freeze/thaw |
| | stability. One set of aliquots at | particular condition tested within | cycles |
| | each level was analyzed on the day | 85-115% recovery. | |
| | of draw, another set was stored at | | |
| | ≤−55° C., and the remaining set was | | |
| | subjected to an additional 6 | | |
| | freeze/thaw. The completed | | |
| | samples from all cycles were | | |
| | stored at ≤−10° C. until analysis. | | |
| | All samples were analyzed in | | |
| | triplicate. Excluding those tested | | |
| | on the same day as preparation, all | | |
| | samples for a given donor were | | |
| | analyzed in a single batch. | | |
| Long-term | Baseline determination for long- | Mean % Recovery from | To be determined |
| Stability | term ≤−10° C. frozen stability will | baseline 85-115% (80-120% at | |
| | be performed as part of the short- | LLOQ), with at least two-thirds | |
| | term and freeze/thaw stability | of the sample replicates at a | |
| | studies. Final measurements to be | particular condition tested within | |
| | completed in a minimum of | 85-115% recovery. | |
| | triplicate in future testing. | | |
| Reference | One hundred and twenty pregnant | Use EP Evaluator or other | Reference Interval: |
| Interval | female patient samples were | appropriate method to verify or | (see above) |
| Verification or | analyzed for reference range | establish reference interval. | |
| Establishment | verification or establishment. | | |
| Selectivity | Potential interferents in presence | Spiked specimen has 85- | Pass for: |
| | of analyte were analyzed. Low | 115% of expected values for | PM3S: 4 of 4 |
| | concentration samples were spiked | analyte | interferents tested |
| | with potential interferents and then | | PM4S: 2 of 3 |
| | analyzed in triplicate. | | interferents tested |
| | | | PM5S: 2 of 3 |
| | | | interferents tested |
| | | | PM2DiS: 3 of 3 |
| | | | interferents tested |
| | | | PM3DiS: 3 of 3 |
| | | | interferents tested |
| Specificity | Potential interferents in absence of | The measured concentration | PM3S, PM4S, |
| | analyte were analyzed. Potential | will be calculated from the | PM5S, PM2DiS, |
| | interferents were spiked into 6% | standard curve. The percent | and PM3DiS |
| | BSA for analysis. | cross-reaction will be calculated | |
| | | as the ratio of the measured | |
| | | concentration to the actual spike | |
| | | concentration of each substance, | |
| | | expressed as a percentage. | |
| | | Insignificant cross-reaction will | |
| | | be defined by a value of <5%. | |
| | | Response < LLOQ | |
| Calibration or | Progesterone Sulfate calibrators. | Minimum of six points per | PM3S, PM4S, |
| Standard Curve | | curve generated. | PM5S, PM2DiS, |
| Precision | | Goodness of fit is | and PM3DiS |
| | | demonstrated by standard curve | |
| | | back-fit calculations. | |
| | | An average variability in | |
| | | concentration of <15% of the | |
| | | expected value is acceptable | |
| | | (20% at LLOQ). | |

Summary

| | | |
|---|---|---|
| Units of measure to report | | ng/mL |
| ULOQ | 500 | ng/mL |
| LLOQ | 1 | ng/mL |

| | |
|---|---|
| AMR (Analytical Measurement Range) | 1-2500 ng/mL for PM3S, PM2DiS and PM3DiS |
| | 1-5000 ng/mL for PM4S and PM5S |
| Max Dilution Limit | X5 (20 uL serum) for PM3S, PM2DiS and PM3DiS |
| | X10 (10 uL serum) for PM4S and PM5S |
| Primary sample type used | Serum |

### Example 3—LC-MS/MS Acquisition Methods

Tables 3-5 show analyst acquisition methods used for PM3S (Table 3), PM4S and PM5S (MPMS) (Table 4), PM2DiS and PM3DiS (PMDiS) (Table 5). Results of such scans are shown in FIG. 4 (PM3S), FIG. 5 (MPMS) and FIG. 6 (PMDiS). As used in Tables 3-5, IS indicates the isotopically labeled standards as described herein. 1, 2 and 3 (as well as sub-designations A, B, C and D) indicate different transitions that are monitored and which may or may not be used for assay quantitation.

TABLE 3
PM3S Acquisition Method

| Analyte | Q1 Mass | Q3 Mass |
|---|---|---|
| PM3S-1 | 399.400 | 97.100 |
| PM3S-2 | 399.400 | 79.700 |
| PM5S-1 | 397.300 | 97.000 |
| PM5S-2 | 397.300 | 79.800 |
| PM4S-1 | 397.200 | 97.000 |
| PM4S-2 | 397.200 | 80.100 |
| PM3S-IS-1 | 403.200 | 98.000 |
| PM3S-IS-2 | 403.200 | 80.000 |
| PM5S-IS-1 | 401.200 | 97.800 |
| PM5S-IS-2 | 401.200 | 80.000 |

TABLE 4
MPMS Analyst Acquisition Method

| Analyte | Q1 Mass | Q3 Mass |
|---|---|---|
| PM3S-1 | 399.400 | 97.100 |
| PM3S-2 | 399.400 | 79.700 |
| PM5S-1 | 397.300 | 97.000 |
| PM5S-2 | 397.300 | 79.800 |
| PM4S-1 | 397.200 | 97.000 |
| PM4S-2 | 397.200 | 80.100 |
| PM3S-IS-1 | 403.200 | 98.000 |
| PM3S-IS-2 | 403.200 | 80.000 |
| PM5S-IS-1 | 401.200 | 97.800 |
| PM5S-IS-2 | 401.200 | 80.000 |

TABLE 5
PMDiS Acquisition Method

| Analyte | Q1 Mass | Q3 Mass |
|---|---|---|
| PMDi - singleCh-1 | 479.101 | 399.100 |
| PMDi - singleCh-1A | 479.100 | 399.100 |
| PMDi - singleCh-1B | 479.099 | 399.100 |
| PMDi - singleCh-1C | 479.098 | 399.100 |
| PMDi - singleCh-1D | 479.102 | 399.100 |
| PMDi - singleCh-1-IS | 483.300 | 403.000 |
| PMDi - singleCh-1A-IS | 483.301 | 403.000 |
| PMDi - singleCh-1B-IS | 483.302 | 403.000 |
| PMDi - singleCh-1C-IS | 483.298 | 403.000 |
| PMDi - singleCh-1D-IS | 483.299 | 403.000 |
| PMDi - singleCh-2 | 479.098 | 381.200 |
| PMDi - singleCh-2A | 479.099 | 381.200 |
| PMDi - singleCh-2B | 479.100 | 381.200 |
| PMDi - singleCh-2C | 479.101 | 381.200 |
| PMDi - singleCh-2D | 479.102 | 381.200 |
| PMDi - singleCh-2-IS | 483.298 | 384.400 |
| PMDi - singleCh-2A-IS | 483.299 | 384.400 |
| PMDi - singleCh-2B-IS | 483.300 | 384.400 |
| PMDi - singleCh-2C-IS | 483.301 | 384.400 |
| PMDi - singleCh-2D-IS | 483.302 | 384.400 |
| PMDi - singleCh-3 | 479.100 | 97.000 |
| PMDi - singleCh-3-IS | 483.300 | 96.900 |

### Example 4—Illustrative Embodiments of Suitable Methods and Systems

As used below, any reference to methods or systems is understood as a reference to each of those methods or systems disjunctively (e.g., “Illustrative embodiment 1-4 is understood as illustrative embodiment 1, 2, 3, or 4.”).

Illustrative embodiment 1 is a method for determining the presence or amount of a progesterone metabolite in a sample from a subject by mass spectrometry comprising the steps of: (a) generating one or more precursor ions from the progesterone metabolite; (b) generating one or more product ions from the one or more precursor ions; (c) detecting the presence or amount of the one or more of the precursor ions generated in step (a) or the one or more product ions of step (b) or both; and relating the detected ions to the presence or amount of the progesterone metabolite in the sample.

Illustrative embodiment 2 is the method of any preceding or subsequent illustrative embodiment, wherein the progesterone metabolite is a progesterone sulfate.

Illustrative embodiment 3 is the method of any preceding or subsequent illustrative embodiment, wherein the progesterone sulfate comprises at least one of 5β-Pregnan-3α, 20α-diol (PM3S); 5α-Pregnan-3α-ol-20-one sulfate (PM4S); 5α-Pregnan-3β-ol-20-one sulfate (PM5S); 5α-Pregnan-3α, 20α-diol disulfate (PM2DiS); or 5β-Pregnan-3α, 20α-diol disulfate (PM3DiS).

Illustrative embodiment 4 is the method of any preceding or subsequent illustrative embodiment, wherein PM3S is measured individually, PM4S and PM5S are measured simultaneously, and PM2DiS and PM3DiS are measured simultaneously.

Illustrative embodiment 5 is the method of any preceding or subsequent illustrative embodiment, wherein the mass spectrometry is tandem mass spectrometry.

Illustrative embodiment 6 is the method of any preceding or subsequent illustrative embodiment, wherein the tandem mass spectrometry is triple quadrupole tandem mass spectrometry.

Illustrative embodiment 7 is the method of any preceding or subsequent illustrative embodiment, wherein selective reaction monitoring (SRM) or multiple reaction monitoring (MRM), optionally with ion summing, is used to select for precursor and/or product ions.

Illustrative embodiment 8 is the method of any preceding or subsequent illustrative embodiment, wherein PM3S is measured by MRM using the analyte peak for the transition of 399.400→97.100.

Illustrative embodiment 9 is the method of any preceding or subsequent illustrative embodiment, wherein PM4S and PM5S are measured by MRM either separately or together using the analyte peak for the 397.3→97.0 transition for PM5S and the analyte peak for the 397.2→97.0 transition for PM4S.

Illustrative embodiment 10 is the method of any preceding or subsequent illustrative embodiment, wherein PM2DiS and/or PM3DiS are measured by MRM using ion summing.

Illustrative embodiment 11 is the method of any preceding or subsequent illustrative embodiment, wherein the sum of 479.098→381.2+479.099→381.2+479.100→381.2+479.101→381.2+479.102→381.2 transitions are used for detection of PM2DiS.

Illustrative embodiment 12 is the method of any preceding or subsequent illustrative embodiment, wherein the sum of 479.098→399.1+479.099→399.1+479.100→399.1+479.101→399.1+479.102→399.1 transitions may be used for detection of PM3DiS.

Illustrative embodiment 13 is the method of any preceding or subsequent illustrative embodiment, wherein the sample is subjected to a purification step prior to the initial fragmentation step (a).

Illustrative embodiment 14 is the method of any preceding or subsequent illustrative embodiment, wherein the purification step comprises chromatography.

Illustrative embodiment 15 is the method of any preceding or subsequent illustrative embodiment, wherein the chromatography comprises high performance liquid chromatography (HPLC) or high throughput liquid chromatography (HTLC).

Illustrative embodiment 16 is the method of any preceding or subsequent illustrative embodiment, further comprising at least one of dilution and/or protein precipitation of the sample prior to chromatography.

Illustrative embodiment 17 is the method of any preceding or subsequent illustrative embodiment, further comprising the addition of stable isotope labeled internal standards.

Illustrative embodiment 18 is the method of any preceding or subsequent illustrative embodiment, wherein the stable isotope labeled internal standards comprise at least one of: PM3S-d₄ (i.e., 5β-Pregnan-3α, 20α-diol-[2,2,4,4-d4] sulfate); PM5S-d₄ (i.e., 5α-Pregnan-3β-ol-20-one-[2,2,4,4-d4] sulfate); PM2DiS-d₄ (i.e., 5α-Pregnan-3α, 20α-diol-[2,2,4,4-d4] disulfate); and/or PM3DiS-d₄ (i.e., 5β-Pregnan-3α, 20α-diol-[2,2,4,4-d4] disulfate).

Illustrative embodiment 19 is the method of any preceding or subsequent illustrative embodiment, wherein the sample is plasma or serum.

Illustrative embodiment 20 is the method of any preceding or subsequent illustrative embodiment, wherein the LLOQ for the progesterone sulfate is 1 ng per 100 μL of the sample.

Illustrative embodiment 21 is the method of any preceding or subsequent illustrative embodiment, wherein the ULOQ for the progesterone sulfate is 500 ng per 100 μL of the sample.

Illustrative embodiment 22 is the method of any preceding or subsequent illustrative embodiment, wherein the amount of the progesterone sulfate is used to distinguish whether gestational pruritus of the skin is an early symptom of (ICP) or due to benign pruritus gravidarum in the subject.

Illustrative embodiment 23 is a system for determining the presence or amount of a progesterone metabolite in a test sample, the system comprising: a station and/or component for providing a test sample suspected of containing a progesterone metabolite of interest; a mass spectrometry station and/or component for fragmentation of the progesterone metabolite of interest to generate a one or more precursor ions and to generate one or more product ions from the one or more precursor ions and to determine the amount of at least one of the precursor ion or the at least one product ion; and a station and/or component to determine the presence or amount of the progesterone metabolite in the test sample.

Illustrative embodiment 24 is the system of any preceding or subsequent illustrative embodiment, wherein the progesterone metabolite of interest is a progesterone sulfate.

Illustrative embodiment 25 is the system of any preceding or subsequent illustrative embodiment, wherein the progesterone sulfate comprises at least one of 5β-Pregnan-3α, 20α-diol (PM3S); 5α-Pregnan-3α-ol-20-one sulfate (PM4S); 5α-Pregnan-3β-ol-20-one sulfate (PM5S); 5α-Pregnan-3α, 20α-diol disulfate (PM2DiS); or 5β-Pregnan-3α, 20α-diol disulfate (PM3DiS).

Illustrative embodiment 26 is the system of any preceding or subsequent illustrative embodiment, wherein PM3S is measured individually (PM3S), PM4S and PM5S are measured simultaneously, and PM2DiS and PM3DiS are measured simultaneously.

Illustrative embodiment 27 is the system of any preceding or subsequent illustrative embodiment, wherein the mass spectrometry is tandem mass spectrometry.

Illustrative embodiment 28 is the system of any preceding or subsequent illustrative embodiment, wherein the tandem mass spectrometry is triple quadrupole tandem mass spectrometry.

Illustrative embodiment 29 is the system of any preceding or subsequent illustrative embodiment, wherein selective reaction monitoring (SRM) or multiple reaction monitoring (MRM), optionally with ion summing, is used to select for precursor and/or product ions.

Illustrative embodiment 30 is the system of any preceding or subsequent illustrative embodiment, wherein PM3S is measured by MRM using the analyte peak for the transition of 399.400→97.100.

Illustrative embodiment 31 is the system of any preceding or subsequent illustrative embodiment, wherein PM4S and PM5S are measured by MRM either separately or together using the analyte peak for the 397.3→97.0 transition for PM5S and the analyte peak for the 397.2→97.0 transition for PM4S.

Illustrative embodiment 32 is the system of any preceding or subsequent illustrative embodiment, wherein PM2DiS and/or PM3DiS are measured by MRM using ion summing.

Illustrative embodiment 33 is the system of any preceding or subsequent illustrative embodiment, wherein the sum of the 479.098→381.2+479.099→381.2+479.100→381.2+479.101→381.2+479.102→381.2 transitions are used for detection of PM2DiS.

Illustrative embodiment 34 is the system of any preceding or subsequent illustrative embodiment, wherein the sum of the 479.098→399.1+479.099→399.1+479.100→399.1+479.101→399.1+479.102→399.1 transitions may be used for detection of PM3DiS.

Illustrative embodiment 35 is the system of any preceding or subsequent illustrative embodiment, further comprising a station and/or component for partially purifying the progesterone metabolite of interest from other components in the sample.

Illustrative embodiment 36 is the system of any preceding or subsequent illustrative embodiment, further comprising a station and/or component for chromatographically separating the progesterone metabolite of interest from other components in the sample.

Illustrative embodiment 37 is the system of any preceding or subsequent illustrative embodiment, wherein the sample is plasma or serum.

Illustrative embodiment 38 is the system of any preceding or subsequent illustrative embodiment, wherein at least one stable isotope is added as an internal standard, and optionally, the stable isotope labeled internal standards comprise at least one of: PM3S-d₄ (i.e., 5β-Pregnan-3α, 20α-diol-[2,2,4,4-d4] sulfate); PM5S-d₄ (i.e., 5α-Pregnan-3β-ol-20-one-[2,2,4,4-d4] sulfate); PM2DiS-d₄ (i.e., 5α-Pregnan-3α, 20α-diol-[2,2,4,4-d4] disulfate); and/or PM3DiS-d₄ (i.e., 5β-Pregnan-3α, 20α-diol-[2,2,4,4-d4] disulfate).

Illustrative embodiment 39 is the system of any preceding or subsequent illustrative embodiment, wherein at least one of the stations and/or components is controlled by a computer and/or a computer-program product tangibly embodied in a non-transitory machine-readable storage medium.

Illustrative embodiment 40 is a computer-program product tangibly embodied in a non-transitory machine-readable storage medium, including instructions configured to perform any of the method steps of illustrative embodiments 1-22 or control any of the stations and/or components of illustrative embodiments 23-39.

Illustrative embodiment 41 is a computer-program product of any preceding or subsequent illustrative embodiment containing instructions which, when executed on one or more data processors, cause one or more data processors to perform actions to direct at least one of the steps of providing a sample believed to contain at least one progesterone metabolite; optionally, chromatographically separating the at least one progesterone metabolite from other components in the sample; using tandem mass spectrometry to generate one or more precursor ions and one or more fragment ions specific to the progesterone metabolite; and determining the presence or amount of the progesterone metabolite in the sample.

Illustrative embodiment 42 is a computer-program product of any preceding or subsequent illustrative embodiment containing instructions which, when executed on one or more data processors, cause the one or more data processors to perform actions to direct at least one of the steps of: (a) generating one or more precursor ions from a progesterone metabolite; (b) generating one or more product ions from the one or more precursor ions; (c) detecting the presence or amount of the one or more of the precursor ions generated in step (a) or the one or more product ions of step (b) or both; and relating the detected ions to the presence or amount of the metabolite of progesterone in the sample.

Illustrative embodiment 43 is the computer-program product of any preceding or subsequent illustrative embodiment containing instructions which, when executed on one or more data processors, cause the one or more data processors to perform actions to run a system or any station and/or component of a system for determining the presence or amount of a progesterone metabolite in a test sample, the system comprising: a station and/or component for providing a test sample suspected of containing a progesterone metabolite of interest; a mass spectrometry station and/or component for fragmentation of the progesterone metabolite of interest to generate one or more precursor ions and to generate one or more product ions from the one or more precursor ions and to determine the amount of the one or more precursor ions or the one or more product ions; and a station and/or component to determine the presence or amount of the progesterone metabolite in the test sample.

Illustrative embodiment 44 is the computer-program product of any preceding or subsequent illustrative embodiment, wherein the progesterone metabolite is a progesterone sulfate.

Illustrative embodiment 45 is the computer-program product of any preceding or subsequent illustrative embodiment, wherein the progesterone sulfate comprises at least one of 5β-Pregnan-3α, 20α-diol (PM3S); 5α-Pregnan-3α-ol-20-one sulfate (PM4S); 5α-Pregnan-3β-ol-20-one sulfate (PM5S); 5α-Pregnan-3α, 20α-diol disulfate (PM2DiS); or 5β-Pregnan-3α, 20α-diol disulfate (PM3DiS).

Illustrative embodiment 46 is the computer-program product of any preceding or subsequent illustrative embodiment, wherein PM3S is measured individually, PM4S and PM5S are measured simultaneously, and PM2DiS and PM3DiS are measured simultaneoulsy measured.

Illustrative embodiment 47 is the computer-program product of any preceding or subsequent illustrative embodiment, wherein the mass spectrometry is tandem mass spectrometry.

Illustrative embodiment 48 is the computer-program product of any preceding or subsequent illustrative embodiment, wherein the tandem mass spectrometry is triple quadrupole tandem mass spectrometry.

Illustrative embodiment 49 is the computer-program product of any preceding or subsequent illustrative embodiment, wherein selective reaction monitoring (SRM) or multiple reaction monitoring (MRM), optionally with ion summing, is used to select for precursor and/or product ions.

Illustrative embodiment 50 is the computer-program product of any preceding or subsequent illustrative embodiment, wherein PM3S is measured by MRM using the analyte peak for the transition of 399.400→97.100.

Illustrative embodiment 51 is the computer-program product of any preceding or subsequent illustrative embodiment, wherein PM4S and PM5S are measured by MRM either separately or together using the analyte peak for the 397.3→97.0 transition for PM5S and the analyte peak for the 397.2→97.0 transition for PM4S.

Illustrative embodiment 52 is the computer-program product of any preceding or subsequent illustrative embodiment, wherein PM2DiS and/or PM3DiS are measured by MRM using ion summing.

Illustrative embodiment 53 is the computer-program product of any preceding or subsequent illustrative embodiment, wherein the sum of 479.098→381.2+479.099→381.2+479.100→381.2+479.101→381.2+479.102→381.2 transitions are used for detection of PM2Di S.

Illustrative embodiment 54 is the computer-program product of any preceding or subsequent illustrative embodiment, wherein the sum of 479.098→399.1+479.099→399.1+479.100→399.1+479.101→399.1+479.102→399.1 transitions may be used for detection of PM3DiS.

Illustrative embodiment 55 is the computer-program product of any preceding or subsequent illustrative embodiment, wherein the sample is subjected to a purification step prior to the initial fragmentation step (a).

Illustrative embodiment 56 is the computer-program product of any preceding or subsequent illustrative embodiment, wherein the purification step comprises chromatography.

Illustrative embodiment 57 is the computer-program product of any preceding or subsequent illustrative embodiment, wherein the chromatography comprises high performance liquid chromatography (HPLC) or high throughput liquid chromatography (HTLC).

Illustrative embodiment 58 is the computer-program product of any preceding or subsequent illustrative embodiment, further comprising at least one of dilution and/or protein precipitation of the sample prior to chromatography.

Illustrative embodiment 59 is the computer-program product of any preceding or subsequent illustrative embodiment, further comprising the addition of stable isotope labeled internal standards.

Illustrative embodiment 60 is the computer-program product of any preceding or subsequent illustrative embodiment, wherein the stable isotope labeled internal standards comprise at least one of: PM3S-d₄ (i.e., 5β-Pregnan-3α, 20α-diol-[2,2,4,4-d4] sulfate); PM5S-d₄ (i.e., 5α-Pregnan-3β-ol-20-one-[2,2,4,4-d4] sulfate); PM2DiS-d₄ (i.e., 5α-Pregnan-3α, 20α-diol-[2,2,4,4-d4] disulfate); and/or PM3DiS-d₄ (i.e., 5β-Pregnan-3α, 20α-diol-[2,2,4,4-d4] disulfate).

Illustrative embodiment 61 is the computer-program product of any preceding or subsequent illustrative embodiment, wherein the sample is plasma or serum.

Illustrative embodiment 62 is the computer-program product of any preceding or subsequent illustrative embodiment, wherein the LLOQ for the progesterone sulfate is 1 ng per 100 μL of the sample.

Illustrative embodiment 63 is the computer-program product of any preceding or subsequent illustrative embodiment, wherein the ULOQ for the progesterone sulfate is 500 ng per 100 μL of the sample.

Illustrative embodiment 64 is the computer-program product of any preceding or subsequent illustrative embodiment, wherein the amount of the progesterone sulfate is used to distinguish whether gestational pruritus of the skin is an early symptom of (ICP) or due to benign pruritus gravidarum in the subject.

Various embodiments of the disclosure have been described herein. It should be recognized that these embodiments are merely illustrative of the present disclosure. Variations of those preferred embodiments may become apparent to those of ordinary skill in the art upon reading the foregoing description. It is expected that skilled artisans can employ such variations as appropriate, and the disclosure is intended to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated or otherwise clearly contradicted by context.

What is claimed is:

1. A method for determining the presence or amount of a progesterone metabolite in a sample from a subject by mass spectrometry comprising the steps of: (a) generating one or more precursor ions from the progesterone metabolite; (b) generating one or more product ions of the one or more precursor ions; (c) detecting the presence or amount of the one or more precursor ions generated in step (a) or the one or more product ions of step (b) or both; and relating the detected ions to the presence or amount of the progesterone metabolite in the sample.

2. The method of claim 1, wherein the progesterone metabolite is a progesterone sulfate.

3. The method of claim 2, wherein the progesterone sulfate comprises at least one of 5β-Pregnan-3α, 20α-diol sulfate (PM3S); 5α-Pregnan-3α-ol-20-one Sulfate (PM4S); 5α-Pregnan-3β-ol-20-one sulfate (PM5S); 5α-Pregnan-3α, 20α-diol disulfate (PM2DiS); or 5β-Pregnan-3α, 20α-diol disulfate (PM3DiS).

4. The method of claim 1, wherein, the mass spectrometry is multiple reaction monitoring tandem mass spectrometry. 5 The method of claim 4, wherein PM3S is measured using a transition of 399.400→97.100; PM5S is measured using a transition of 397.3→97.0 transition; PM4S is measured using a transition of 397.2→97.0; PM2DiS is measured using the sum of transitions at 479.098→381.2+479.099→381.2+479.100→381.2+479.101→381.2+479.102→381.2; and PM3DiS is measured using the sum of transitions at transitions at 479.098→399.1+479.099→399.1+479.100→399.1+479.101→399.1+479.102→399.1.

6. The method of claim 1, wherein the sample is subjected to a purification step prior to mass spectrometry.

7. The method of claim 6, wherein the purification step comprises high performance liquid chromatography (HPLC).

8. The method of claim 6, wherein the purification step comprises at least one of dilution and/or protein precipitation of the sample.

9. The method of claim 3, wherein PM3S is measured individually, PM4S and PM5S are measured simultaneously, and PM2DiS and PM3DiS are measured simultaneoulsy.

10. The method of claim 1, further comprising the addition of stable isotope labeled internal standards.

11. The method of claim 10, wherein the stable isotope labeled internal standards comprise at least one of 5β-Pregnan-3α, 20α-diol-[2,2,4,4-d4] sulfate (PM3S-d₄), 5α-Pregnan-3β-ol-20-one-[2,2,4,4-d4] sulfate (PM5S-d₄), 5α-Pregnan-3α, 20α-diol-[2,2,4,4-d4] disulfate (PM2DiS-d₄), or 5β-Pregnan-3α, 20α-diol-[2,2,4,4-d4] disulfate (PM3DiS-d₄).

12. The method of claim 1, wherein the sample is human serum or plasma.

13. The method of claim 2, wherein the lower limit of quantitation (LLOQ) for the progesterone sulfate is 1 ng per 100 μL of the sample.

14. The method of claim 2, wherein the upper limit of quantitation (ULOQ) for the progesterone sulfate is 500 ng per 100 μL of the sample.

15. The method of claim 1, wherein amount of progesterone sulfate is used to distinguish whether gestational pruritus of the skin is an early symptom of (ICP) or due to benign pruritus gravidarum in the subject.

16. A system for determining the presence or amount of a progesterone metabolite in a test sample from a subject, the system comprising:
a station and/or component for providing a test sample suspected of containing a progesterone metabolite of interest;
a station and/or component for mass spectrometry for fragmentation of the progesterone metabolite of interest to generate one or more precursor ions and one or more product ions; and a station and/or component to determine the presence or amount of the progesterone metabolite of interest in the sample.

17. The system of claim 16, wherein the progesterone metabolite of interest is a progesterone sulfate.

18. The system of claim 16, further comprising a station and/or component for partially purifying the progesterone metabolite of interest from other components in the sample.

19. The system of claim 17, further comprising a station and/or component for chromatographically separating the progesterone metabolite of interest from other components in the sample.

20. A computer-program product which, when executed on one or more data processors, cause the one or more data processors to perform actions to direct at least one of the steps of: (a) generating one or more precursor ions from a progesterone metabolite; (b) generating one or more product ions of the one or more precursor ions; (c) detecting the presence or amount of one or more of the precursor ions generated in step (a) or the one or more product ions of step (b) or both; and relating the detected ions to the presence or amount of the metabolite of progesterone in the sample.
